(19) United States
(12) Patent Application Publication
(10) Pub. No.: US 2024/0319213 A1
(43) Pub. Date: Sep. 26, 2024

(54) SCREENING DONOR LUNGS FOR LUNG TRANSPLANTATION

(71) Applicant: UNIVERSITY HEALTH NETWORK, Toronto (CA)

(72) Inventors: Shafique Keshavjee, Toronto (CA); Andrew Sage, Toronto (CA); Marcelo Cypel, Toronto (CA); Tereza Martinu, Toronto (CA)

(21) Appl. No.: 18/694,171

(22) PCT Filed: Sep. 26, 2022
(86) PCT No.: PCT/CA2022/051424

§ 371 Date: Mar. 21, 2024

Related U.S. Application Data
(60) Provisional application No. 63/248,226, filed on Sep. 24, 2021.

(51) Int. Cl. G01N 33/92; G01N 33/543; G01N 33/68

(52) U.S. Cl. CPC G01N 33/92; G01N 33/54388; G01N 33/6869; G01N 33/6893; G01N 2333/521; G01N 2333/5412; G01N 2333/5421; G01N 2800/06; G01N 2800/24; G01N 2800/50

(57) ABSTRACT

Methods, kits and devices for assessing bile acid in a donor lung and/or a transplant recipient are described. The methods involve obtaining from the donor lung or transplant recipient a bronchial wash sample, optionally a bronchoalveolar lavage (BAL) sample or a large airway bronchial wash (LABW) sample; measuring in the bronchial wash sample the level of bile acid and optionally one or more inflammation markers, comparing biomarker levels with a control or cut-off level, wherein a differential biomarker level is indicative of an outcome of the donor lung or transplant recipient, including risk of aspiration, suitability of the donor lung, or risk of a particular outcome in the transplant recipient.

### RELATED APPLICATION

This disclosure claims benefit and priority of U.S. Provisional Patent Application Ser. No. 63/248,226 filed Sep. 24, 2021, which is incorporated herein by reference in its entirety.

### FIELD

The disclosure pertains to methods, kits and devices for detecting bile acid in a donor lungs and for assessing the suitability of donor lungs for transplantation.

### BACKGROUND

Despite the increasing number of lung transplants each year, approximately 75 to 80% of all donor lungs are declined for transplantation (1-3). The selection of the healthiest donor lungs is based on a number of clinical findings that include: donor type (brain vs cardiac death donors), age, smoking, ABGs, chest radiograph and bronchoscopic findings, and physical examination by the physician (1-3). One reason for rejecting donor lungs is the clinical suspicion of aspiration (4-5). Aspiration arises when gastric contents from the stomach that enter the lung (4-5). Aspiration can also lead to cell damage, death, and an advanced inflammatory response (4-5).

Ex vivo lung perfusion (EVLP) is a recent technique that was developed to prolong the normothermic assessment period of donor organs during lung transplantation (6-10). EVLP has been clinically validated and has gained widespread adoption worldwide. One of the indications for EVLP is the suspicion of aspiration in the donor, prior to organ retrieval. However, current assessment of aspiration mainly relies on bronchoscopy which only confirms macro aspiration.

Following transplantation, gastroesophageal reflux disease (GERD) is a condition where patients experience chronic gastrointestinal content backwash to the esophagus. Previous research has illustrated that lung transplantation could increase GERD in patients (12). GERD is known to cause microaspiration which exposes the lung to gastric contents and leads to chronic inflammation of lung tissues (13). Consequently, GERD has been considered as a risk factor for chronic lung allograft dysfunction (CLAD), hindering long-term survival in lung transplant patients (14). The current strategies for diagnosing GERD rely on the ambulatory pH monitoring in the distal esophagus during a 24-hr window (15). The limitations of pH monitoring include patient discomfort, limited time of observation and poor patient compliance.

The development of technologies such as rapid point-of-care technologies is desirable in order to enable for example a more accurate detection of aspiration and aid in the clinical decision-making processes for lung transplant patients.

### SUMMARY

The inventors have identified bile acid levels that are associated with significant aspiration in a donor lung and with suitability of the donor lung for transplant. The inventors also identified bile acid levels that are associated with GERD in donor lung recipients.

The disclosure provides in an aspect a method of assessing significant aspiration in a donor lung, the method comprising:
a) obtaining a bronchial wash sample that has been obtained from the donor lung;
b) measuring a level of a bile acid in the bronchial wash sample;
c) comparing the level of the bile acid with a bile acid cut-off level; and
d) identifying the donor lung as likely to have had significant aspiration when the level of the bile acid is greater than the bile acid cut-off level, or unlikely to have had significant aspiration when the bile acid level is equal to or less than the bile acid cut-off level, optionally wherein the bile acid cut-off level is determined by calculating in a population of donor lungs an average bile acid cut-off level above which at least 90% of the donor lungs were identified as having significant aspiration.

Another aspect herein provided is a method of assessing suitability of a donor lung for transplant, the method comprising:
a) obtaining a bronchial wash sample that has been obtained from the donor lung;
b) measuring a level of a bile acid in the bronchial wash sample and optionally a level of one or more inflammation markers;
c) comparing the level of the bile acid with a bile acid cut-off level,
d) optionally comparing the level of the one or more inflammation markers with an inflammation marker cut-off level;
e) identifying the donor lung as not suitable for transplant when the level of the bile acid is greater than the bile acid cut-off level, and optionally when the level of the one or more inflammation markers is greater than the inflammation marker cut-off level, or as suitable for transplant when the level of the bile acid is equal or less than the bile acid cut-off level, and optionally when the level of the one or more inflammation markers is equal or less than the inflammation marker cut-off level, optionally wherein the bile acid cut-off level is determined by calculating in a population of donor lungs an average bile acid cut-off level above which at least 90% of the donor lungs were identified as not suitable for transplantation and the inflammation marker cut-off level is determined by calculating in a population of donor lungs an average inflammation marker cut-off level above which at least 90% of the donor lungs were identified as not suitable for transplantation.

Another aspect relates to a method of assessing the likelihood of developing gastroesophageal reflux disease (GERD) in a donor lung recipient, the method comprising:
a) obtaining a bronchial wash sample from the donor lung recipient;
b) measuring a level of a bile acid in the bronchial wash sample; and
c) comparing the level of the bile acid with a bile acid cut-off level, and
d) identifying the donor lung recipient as likely to develop GERD when the level of the bile acid is greater than the bile acid cut-off level, or unlikely to develop GERD when the bile acid level is equal to or less than the bile acid cut-off level, optionally wherein the bile acid cut-off level is determined by calculating in a population of donor lungs an average bile acid cut-off level above which at least 90% of the donor lungs were identified as having developed GERD.

In an embodiment, when the level of the bile acid is greater than the bile acid cut-off level, the method described herein further comprises performing EVLP to confirm lung function prior to transplantation.

In an embodiment, when the level of the bile acid is greater than the bile acid cut-off level, the method described herein further comprises treating the lung for aspiration damage. In an embodiment, the method comprises treating the lung with surfactant.

In an embodiment, when the level of the bile acid is greater than the bile acid cut-off level, the method described herein further comprises rejecting the lung for transplantation.

In an embodiment, the bronchial wash sample is a bronchoalveolar lavage (BAL) sample or a large airway bronchial wash (LABW) sample.

In an embodiment, the bile acid is total bile acid (TBA) or a component thereof optionally selected from taurocholic acid (TCA), glycocholic acid (GCA) and cholic acid (CA).

In an embodiment, the bile acid is TBA.

In an embodiment, the bile acid and optionally the one or more inflammation markers are detected or determined by Luminex® based assays, Western blots, immunoassay e.g. ELISA, immunofluorescence, radioimmunoassay, dot blotting, FACS, protein microarray, immunoprecipitation followed by SDS-PAGE, immunocytochemistry, simple Plex assay, multiplex assay, mass spectrometry, electrochemical assay, enzymatic assay or colorimetric assay.

The disclosure also includes assays, devices and kits for the detection of the bile acids and optionally inflammation markers.

An aspect herein disclosed is a kit comprising at least one detection agent specific for a biomarker selected from bile acid, optionally total bile acid, taurocholic acid (TCA), glycocholic acid (GCA) or cholic acid (CA), and optionally an inflammation marker, optionally interleukin-6 (IL-6), interleukin-8 (IL-8), interleukin-1 alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10), the kit optionally further comprising one or more of a 96-well plate, standards, assay buffer, wash buffer, sample diluent, standard diluent, detection antibody diluent, streptavidin-PE, a filter plate and sealing tape, optionally for performing the methods herein disclosed.

A point-of-care (POC) device for detecting the presence of bile acid in a sample, comprising:
a housing defining:
   a sample portion for receiving a fluid sample and a testing portion,
   a porous membrane connecting the sample portion to the testing portion, the porous membrane configured to draw the fluid sample from the sample portion to the testing portion; and
   one or more reagents or detection agents for bile acid immobilized on the testing portion; wherein the one or more reagents or detection agents are capable of reacting with the bile acid to generate a detection signal.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the disclosure are described with reference to the drawings:

FIG. 1—Total bile acid (TBA) measured in large airway bronchial wash (LABW) of donor lungs. FIG. 1A is a diagram of different groups of study participants.

FIG. 1B is a graph showing LABW TBA in donor lungs rejected (n=42), transplanted (n=350), or assessed on ex vivo lung perfusion (EVLP) (n=213). FIG. 1C is a graph comparing TBA in donor lungs that were rejected (n=56) vs transplanted (n=157) after assessment on EVLP. P-values were calculated using Wilcoxon-Mann-Whitney test.

FIG. 2—Associations between donor LABW TBA with markers of lung biochemistry and physiology assessed in EVLP perfusate. FIG. 2A is a heatmap of association between donor LABW TBA with electrolytes, markers of acid/base chemistry, and inflammatory mediators measured at different times in EVLP perfusate (n=213). Shading represents direction and magnitude of Spearman correlation coefficient. P-values were calculated using Spearman correlation. Physiology of donor lungs assessed at each hour on EVLP in lungs with high (n=21) vs low (n=192) LABW TBA. [Abbreviations: GM-CSF-granulocyte-macrophage colony stimulating factor; IL-interleukin; sTNFR1-soluble tumour necrosis factor a receptor 1; sTREM1-soluble triggering receptor expressed on myeloid cells 1; 1 hour—measured in perfusate collected after one hour on EVLP; Last-measured in perfusate collected at the last hour before organ was removed from the circuit; Average—average of measurements in EVLP perfusate collected every hour organs were on the circuit; PVR—pulmonary vascular resistance. FIG. 2B is a graph comparing Delta PO₂ measured at each hour on EVLP in donor lungs with high (right bar) vs low (left bar) TBA, FIG. 2C is a graph comparing PVR measured at each hour on EVLP in donor lungs with high vs low TBA. FIG. 2D is a graph comparing static compliance measured at each hour on EVLP in donor lungs with high vs low TBA. FIG. 2E is a graph comparing dynamic compliance measured at each hour on EVLP in donor lungs with high vs low TBA. P-values were calculated by comparing the two groups at every time point using Wilcoxon-Mann-Whitney test. *p<0.05; **p<0.01; ***p<0.001; ns—not significant.

FIG. 3A is a receiver operator characteristic curve modelling LABW TBA concentration and lungs directly transplanted vs directly rejected for transplant. FIG. 3B is a diagram showing prevalence of donor TBA level in different groups of lung donors.

FIG. 4 is a series of Kaplan-Meier curves for time to extubation (FIG. 4A) and ICU discharge (FIG. 4B) as well as cumulative risk of chronic lung allograft dysfunction (CLAD) (FIG. 4C) or death in donors (FIG. 4D) with high vs low LABW total bile acid (TBA) level. CLAD was defined as per the International Society for Heart and Lung Transplantation consensus report (Verleden et al. JHLT 2019). TBA high-TBA>1245 nM; TBA low-TBA<1245 nM. P-values were calculated using log-rank comparisons.

FIG. 5 is a series of graphs comparing donor lungs with high TBA level that were directly transplanted (n=28) vs donor lungs assessed on EVLP before transplant (n=21). FIG. 5A is a graph showing LABW TBA level of donor lungs with TBA>1245 nM that were directly transplanted vs assessed on EVLP before transplant. P-value was calculated using Wilcoxon-Mann-Whitney test. Kaplan-Meier curves for time to extubation (FIG. 5B), time to ICU discharge (FIG. 5C), time to CLAD (FIG. 5D), and time to death (FIG. 5E) among donor lungs with TBA>1245 nM that were directly transplanted vs assessed on EVLP before transplant.

FIG. 6 is a series of graphs showing improvement assessment of donor lungs by combining LABW TBA level with Toronto Lung Score (TLS₂). FIG. 6A is a graph grouping donor lungs based on LABW TBA level and TLS₂. Group 1 lungs (top left quadrant) had both low TBA and favourable TLS₂, Groups 2+3 lungs (top right and bottom left quadrants) had either high TBA or poor TLS₂ but not both, Group 4 (bottom right quadrant) had both high TBA and poor TLS₂. Different groups of donor lungs were compared for their respective recipients' time to extubation (FIG. 6B) and time to ICU discharge (FIG. 6C). P-values were calculated using Wilcoxon-Mann-Whitney test. Recipients of different groups of donor lungs were compared for time to chronic lung allograft dysfunction (FIG. 6D) as well as time to death or retransplant (FIG. 6E). P-values were calculated using log-rank comparisons. *p<0.05; **p<0.01; ***p<0.001; ns—not significant.

FIG. 7 is a graph showing differences in groups using ELISA. TBA was detectable in 43% of lungs that were not accepted for transplant, 2% of lungs that were directly transplanted, and 30% of lungs that were further assessed on EVLP (p-value<0.001).

FIG. 8 is a graph showing the results relating to primary graft dysfunction (PGD) at admission using ELISA. The results indicate a trend towards more prevalent PGD3 in patients with detectable TBA.

FIG. 9 is a graph showing the results relating primary graft dysfunction (PGD) at 72 h post-operation using ELISA. The results indicate a trend towards more prevalent PGD3 in patients with detectable TBA.

### DETAILED DESCRIPTION OF THE DISCLOSURE

### I. Definitions

The term “bronchial wash” as used herein means a liquid obtained by washing (or lavage) of lung airway(s). The washing comprises instilling and retrieving an amount of liquid (e.g. saline) through a bronchial airway for analysis. For example, the bronchial wash can be a large airway bronchial wash (LABW) or a broncoalveolar lavage (BAL). Bronchial wash samples can be used to assess lung pathology and physiology. For example, as shown herein bronchial wash samples can be used to assess levels of bile acid and other biomarkers such as inflammation markers. A bronchial wash sample can be obtained from a donor lung as well as from a patient for example after a patient has been transplanted with a donor lung using known techniques.

The term “aspiration” as used herein refers to a condition where oral content or gastric content from the stomach enters the lung. Bile acids, for example total bile acid, in bronchial wash can be measured to assess aspiration in the lung. The presence of gastric content such as bile acid can damage lung tissue and impair physiological performance of the lung. Aspiration can also lead to cell damage, death and advanced inflammatory response.

The term “significant aspiration” as used herein means a level of aspiration in a lung that renders the lung unsuitable for transplantation. A lung may be unsuitable for transplant if for example PaO₂ levels are not suitable, if airway pressure is not suitable or if the lung the lung tissue is sufficiently damaged. PaO₂ is a metric in the donor lung for determining organ suitability for transplantation. Some other physiological features useful in determining suitability for transplantation include lung compliance and airway pressure(s). Aspiration can be assessed by measuring a level of a bile acid in a bronchial wash sample. A cut-off level of bile acid can be selected according to for example a desired level of risk or particular patient characteristics and used to identify if a donor lungs has had significant aspiration and therefore is unsuitable for transplantation.

The term “point-of-care” or “POC” as used herein means that testing using a method or device is performed or used near the potential lung donor or near the lung transplant recipient. For example, the testing does not have to be performed in a dedicated facility such as a pathology laboratory which may require trained technicians and professionals. “Point-of-care” testing can be accomplished through the use of transportable, portable and/or handheld devices and test kits (e.g. a point of care device). Advantages of point-of-care testing include the ability to obtain rapid analytical results, especially in emergency and/or time constrained situations. “Point-of-care devices” include, but are not limited to, lateral flow devices, lab-on-a child technologies, dipstick assay devices and colorimetric tests.

The term “biomarker” as used herein refers to one or more bile acids and optionally one or more inflammation markers. The term biomarker can also refer to a combination of bile acids, such as any combination of bile acids described herein or to TBA.

The term “EVLP naïve” as used herein means a donor lung that has not been subjected to EVLP.

The term “bile acid” as used herein means total bile acid (TBA) or a component thereof for example selected from taurocholic acid (TCA), glycocholic acid (GCA) and cholic acid (CA). As it relates to TBA assay, TBA refers to the detection of common/conserved elements of any/all bile acids. A TBA assay can report levels of all bile acids, whether conjugated and unconjugated, as one value. The assay may also report levels individually. As used herein, “bile acid” includes conjugated and unconjugated bile acid. Conjugated bile acid is known in the art, for example TCA is a conjugated bile acid.

The term “inflammation marker” as used herein includes interleukin-6 (IL6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10).

The term “IL-6” or “IL6” as used herein means interleukin-6 which is a secreted cytokine, and includes all naturally occurring forms, for example from all species and particularly human including for example human IL-6 which has amino acid sequence accession P05231, herein incorporated by reference.

The term “IL-8” also referred to as CXCL8, as used herein means interleukin-8 which is a secreted cytokine, and includes all naturally occurring forms, for example from all species and particularly human including for example human IL-8 which has amino acid sequence accession P10145, herein incorporated by reference.

The term “polypeptide” as used herein refers to a polymer consisting of a number of amino acid residues bonded together in a chain. The polypeptide can form a part or the whole of a protein. The polypeptide can be arranged in a long, continuous and unbranched peptide chain. The polypeptide can also be arranged in a biologically functional way. The polypeptide can be folded into a specific three dimensional structure that confers it a defined activity. The term “polypeptide” as used herein is used interchangeably with the term “protein”.

The term “cut-off level” as used herein refers a predetermined threshold value based on one or a plurality of known patient recipient outcomes, and with respect to biomarkers associated with increased level in poor grafts e.g. those identified as likely to have aspiration and not being suitable for transplant (e.g. rejected, further assessed with EVLP, declined following EVLP), above which threshold a graft is identified as having an increased risk of developing negative outcome post-transplant and/or being declined after EVLP and below which (and/or comparable to) a candidate donor lung is identified as having a decreased risk of developing poor outcome post-transplant or being declined post EVLP. The threshold value can for example for each of the one or more biomarkers herein described, be determined from the levels or parameter values related thereto of the biomarkers in a plurality of known outcome lungs. For example, an optimal or an acceptable threshold can be selected based on the desired tolerable level of risk. The cut-off level can for example depend on the outcome being assessed. For example, for the patient outcome “ICU stay”, patients that have pre-transplant ICU stay are typically sicker and would also have longer post-transplant ICU stays. The cut-off level can be, for example, a level associated with duration of mechanical ventilation less than 72 h as a threshold in the ICU. The cut-off level can also for example be a value that is adjusted for or includes donor characteristics, for example gender, type (DBD or DCD), age, body mass index (BMI), and/or smoking history. Accordingly, the biomarker ‘cut-off’ can in some embodiments be adjusted for patients who have different duration of ICU stays, and donor characteristics. In an embodiment, the threshold is 72 h in the ICU. Multiple cut-off levels can also be employed. For example, there can be a first (low), second (medium) and third (high) cut off level. The “high” cut-off level, can for example be a predetermined level that is determined by calculating from a population that has clearly significant aspiration and identified as not suitable for transplantation. of donor lungs an average inflammation marker cut-off level above which at least 90% of the donor lungs were identified as not suitable for transplantation. The medium cut-off level can for example be a predetermined level that is determined by calculating from a population that has average or an intermediate level of aspiration and potentially suitable for transplantation, for example in recipients that are less sick. The low cut-off level can be for example a predetermined level that is determined by calculating from a population that has slight or no aspiration and which lung would be suitable in most (e.g. greater than 90%) or all patients . . .

The term “suitable for transplant” as used herein means a lung that is predicted to be a good patient outcome lung graft, for example decreased risk of a prolonged ICU stay post-transplant, decreased time to extubation and decreased risk of developing CLAD, as compared to, for example, lungs with unsuitable bile acid and/or inflammation biomarker levels (e.g. as determined based on a population of lung transplant outcome and biomarker data). Levels of bile acid and optionally inflammation markers can be used to assess whether the donor lung is suitable for transplant. For example, when a biomarker level from the donor lung (e.g. bile acid such as TBA) is equal to or less than a pre-determined cut-off level, the donor lung is identified as suitable for transplant. “Suitable for transplant” means that the donor lung can be directly transplanted (e.g. without subjecting to EVLP) to a donor lung recipient. Alternatively, the donor lung can be further assessed by being subjected to EVLP and repeating assessment of the levels of bile acid and optionally inflammation markers.

The term “unsuitable for transplant” as used herein means a lung that is predicted to be a negative or poor patient outcome lung graft, for example increased risk of prolonged ICU stay post-transplant, increased time to extubation and increased risk of developing CLAD as compared to, for example, lungs with low bile acid and/or inflammation biomarker levels. Levels of bile acid and optionally inflammation markers can be used to assess whether the donor lung is unsuitable for transplant. For example, when a biomarker level from the donor lung (e.g. bile acid such as TBA) is greater than a pre-determined cut-off level, the donor lung is identified as unsuitable for transplant. Unsuitable lungs are either declined or can be further assessed by being subjected to EVLP.

The term “declined lungs” as used herein means lungs that after assessment and/or EVLP are declined for transplant. Such lungs can be discarded. Lungs are presently typically declined for example if gas exchange function is not acceptable, represented by a partial pressure of oxygen less than 350 mmHg with a fraction of inspired oxygen of 100%; or 15% worsening of lung compliance (represented by standard lung metric) compared to 1 h EVLP; or 15% worsening of pulmonary vascular resistance compared to 1 h EVLP; or development of significant edema; or worsening of ex vivo x-ray. In the present methods, lungs can be declined even if the gas exchange function is acceptable, there is no worsening of lung compliance or pulmonary vascular resistance, lack of edema or stable ex vivo x-ray if, for example, the bile acids are above an acceptable cut off level. Declining compliance indicates an injured lung; stable compliance is good and improves likelihood lung would be transplanted.

The term “PGD3” as used herein means Primary Graft Dysfunction Grade 3 as defined by the standardized consensus criteria of International Society for Heart and Lung Transplantation (ISHLT) or similar.

The term “antibody” as used herein is intended to include monoclonal antibodies including chimeric and humanized monoclonal antibodies, polyclonal antibodies, humanized antibodies, human antibodies, and chimeric antibodies. The antibody can be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. The skilled person can readily recognize that a suitable antibody is any antibody useful for detecting biomarkers described herein in any detection method described herein. For example, useful antibodies include antibodies that specifically bind to bile acids (e.g. TCA and GCA), IL-6 or IL-8 polypeptide.

The term “detection agent” refers to an agent (optionally a detection antibody) that selectively binds and is capable of binding its cognate biomarker compared to another molecule and which can be used to detect a level and/or the presence of the biomarker. A biomarker specific detection agent can include probes and the like as well as binding polypeptides such as antibodies which can for example be used with Luminex® based assays, ELISA, immunofluorescence, radioimmunoassay, dot blotting, FACS, protein microarray, Western blots, immunoprecipitation followed by SDS-PAGE immunocytochemistry Simple Plex assay or Mass Spectrometry to detect the polypeptide level of a biomarker described herein. Similarly, “an antibody or fragment thereof” (e.g. binding fragment), that specifically binds a biomarker refers to an antibody or fragment that selectively binds its cognate biomarker compared to another molecule. “Selective” is used contextually, to characterize the binding properties of an antibody. An antibody that binds specifically or selectively to a given biomarker or epitope thereof can bind to that biomarker and/or epitope either with greater avidity or with more specificity, relative to other, different molecules. For example, the antibody can bind 3-5, 5-7, 7-10, 10-15, 5-15, or 5-30 fold more efficiently to its cognate biomarker compared to another molecule. The “detection agent” can for example be coupled to or labeled with a detectable marker. The label is preferably capable of producing, either directly or indirectly, a detectable signal. For example, the label can be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The term “level” as used herein refers to an amount (e.g. relative amount or concentration as well as parameter values calculable based thereon such as a rate or ratio) of biomarker (i.e. polypeptide related level) that is detectable, measurable or quantifiable in a test biological sample and/or a reference biological sample, for example, a BAL sample and/or a reference BAL sample. For example, the level can be a rate such as pg/mL/hour, a concentration such as μg/L, ng/mL or pg/mL, a relative amount or ratio such as 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, and/or 30 times more or less than a control biomarker or reference profile level. The control biomarker polypeptide level can, for example, be the average or median level in a plurality of known outcome lungs. Parameter values related to a level include concentration, rate of production and a ratio or fold increase (e.g. concentration at a later time point (such as 4 hours) divided by a concentration at an earlier time point for the same biomarker).

The term “subject” as used herein includes all members of the animal kingdom including mammals, and suitably refers to humans.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined can be combined with any other aspect or aspects unless clearly indicated to the contrary. In particular, any feature indicated as being preferred or advantageous can be combined with any other feature or features indicated as being preferred or advantageous.

### II. Methods

Disclosed herein are bile acid and inflammation markers that can be used to assess whether a donor lung has aspiration, whether the donor lung is suitable for transplant and/or whether a donor lung recipient is at increased risk of GERD or other outcomes. More particularly, these biomarkers are measured from bronchial wash samples such as large airway bronchial wash sample and bronchoalveolar lavages samples. Such biomarkers can be used as a point-of-care (POC) screening tool, facilitating early triage of donor lungs and allowing surgeons to better assess which lungs are suitable to go directly to transplantation, those that will require further assessment using EVLP and those not suitable for transplantation. POC devices herein disclosed can also help in determining the occurrence of pulmonary aspiration, which can enable early diagnostic and management of GERD post-transplant to minimize allograft injury and chronic lung inflammation. The methods disclosed herein can be qualitative, semi-quantitative, or quantitative. For example, qualitative evaluation can be a colorimetric assay where the result can be assessed visually, as in the detection of the presence or absence of the line in a pregnancy test. Semi-quantitative evaluation can, for example, based on the number and the intensity of multiple test lines (see e.g., Parolo C et al., Tutorial: design and fabrication of nanoparticle-based lateral-flow immunoassays. Nat Protoc 15, 3788-3816 (2020), the content of which is incorporated by reference herein in its entirety).

One aspect of the present disclosure is a method of assessing significant aspiration in a donor lung, the method comprising:
a) obtaining a bronchial wash sample that has been obtained from the donor lung;
b) measuring a level of a bile acid in the bronchial wash sample;
c) comparing the level of the bile acid with a bile acid cut-off level; and
d) identifying the donor lung as likely to have had significant aspiration when the level of the bile acid is greater than the bile acid cut-off level or unlikely to have had significant aspiration when the bile acid level is equal to or less than the bile acid cut-off level, optionally wherein the bile acid cut-off level is determined by calculating in a population of donor lungs an average bile acid cut-off level above which at least 90% of the donor lungs were identified as having significant aspiration.

In an embodiment, when the level of the bile acid is greater than the bile acid cut-off level, the method further comprises performing EVLP to confirm lung function prior to transplantation.

In an embodiment, when the level of the bile acid is greater than the bile acid cut-off level, the method further comprises treating the lung for aspiration damage. In an embodiment, the method comprises treating the lung with surfactant.

In an embodiment, when the level of the bile acid is greater than the bile acid cut-off level, the method further comprises rejecting the lung for transplantation.

The method may comprise further assessing the lung after performing EVLP or after treating the lung for aspiration damage. The assessment post EVLP or treatment may be assessing for bile acids, such as TBAs.

Other parameters can also be assessed. For example, in an embodiment, the PaO₂ is assessed. In another embodiment, the lung compliance is assessed. In an embodiment, the airway pressure is assessed. These parameters can be assessed initially or after EVLP or treatment.

In an embodiment, the bronchial wash sample is a BAL sample. In an embodiment, the bronchial wash sample is a LABW sample.

The sample may be centrifuged and the supernatant assessed, optionally directly (e.g. without concentration). The supernatant or sample may be frozen. The supernatant or sample may be concentrated.

In an embodiment, the bronchial wash sample is a BAL sample obtained in accordance with the International Society for Heart and Lung Transplantation Consensus Statement for the Standardization of Bronchoalveolar Lavage in Lung Transplantation (Martinu et al. J Heart Lung Transplant, “International Society for Heart and Lung Transplantation consensus statement for the standardization of bronchoalveolar lavage in lung transplantation”, 2020 November; 39(11):1171-1190; PMID: 32773322).

In an embodiment, the bronchial wash sample is obtained before retrieval (i.e. while the lung is still in the body of the donor). In an embodiment, the bronchial wash sample is obtained shortly after retrieval from donor, for example 0.5, 1, 2, 4, 6, 8, 10 or 12 hours following retrieval. In some embodiments, the bronchial wash sample is obtained before EVLP or during EVLP. In some embodiments, the bronchial wash sample is obtained after 0.5, 1, 2, 3, 4, 5 or 6 hours of EVLP.

In another embodiment, the bile acid is selected from one or more of taurocholic acid (TCA), glycocholic acid (GCA), cholic acid (CA), and combinations thereof.

In an embodiment, the bile acid is total bile acid (TBA).

In an embodiment, the cut-off level (TBA cut-off level) is about 1245 nM and a level of the TBA greater than the cut-off level is indicative the donor lung as likely to have significant aspiration and a level of TBA equal to or less than the cut-off level is indicative the donor lung is unlikely to have had significant aspiration, wherein the cut-off level is calculated by instilling and/or suctioning about 20 mL of normal saline. The skilled person recognizes that the volume suctioned is less than the volume instilled, because some of the fluid (e.g. 10%, 15% or 20%) gets lost in the airways. Different cut off levels can be calculated for different volumes. Different cut off levels can be calculated depending on how the sample or supernatant is processed. In an embodiment the cut off level is about 1250 nM,

In an embodiment, the method is carried out at point-of-care.

A further aspect relates to a method of assessing suitability of a donor lung for transplant, the method comprising:
a) obtaining a bronchial wash sample that has been obtained from the donor lung;
b) measuring a level of a bile acid in the bronchial wash sample and optionally a level of one or more inflammation markers;
c) comparing the level of the bile acid with a bile acid cut-off level,
d) optionally comparing the level of the one or more inflammation markers with an inflammation marker cut-off level;
e) identifying the donor lung as not suitable for transplant when the level of the bile acid is greater than the bile acid cut-off level, and optionally when the level of the one or more inflammation markers is greater than the inflammation marker cut-off level, or as suitable for transplant when the level of the bile acid is equal or less than the bile acid cut-off level, and optionally when the level of the one or more inflammation markers is equal or less than the inflammation marker cut-off level, optionally wherein the bile acid cut-off level is determined by calculating in a population of donor lungs an average bile acid cut-off level above which at least 90% of the donor lungs were identified as not suitable for transplantation and the inflammation marker cut-off level is determined by calculating in a population of donor lungs an average inflammation marker cut-off level above which at least 90% of the donor lungs were identified as not suitable for transplantation.

In an embodiment, when the level of the bile acid is greater than the bile acid cut-off level, the method described herein further comprises performing EVLP to confirm lung function prior to transplantation.

In an embodiment, when the level of the bile acid is greater than the bile acid cut-off level, the method described herein further comprises treating the lungs for aspiration damage. In an embodiment, the method comprises treating the lungs with surfactant.

In an embodiment, when the level of the bile acid is greater than the bile acid cut-off level, the method described herein further comprises rejecting the lung for transplantation.

In an embodiment, the bronchial wash sample is a BAL sample. In an embodiment, the bronchial wash sample is a LABW sample.

In another embodiment, the bile acid is selected from one or more of taurocholic acid (TCA), glycocholic acid (GCA), cholic acid (CA), and combinations thereof.

In an embodiment, the bile acid is total bile acid (TBA).

As shown herein, the inventors have identified that a TBA cut-off of about 1245 nM (using 20 mL) was able to identify donor lungs unsuitable for transplant with 91% specificity.

In an embodiment, the method involves comparing to a cut-off. For example, each marker can have a different cut-off depending on statistical calculations and/or desired test sensitivity and/or specificity. Where more than one biomarker is assessed, a composite score can be determined.

In an embodiment, the cut-off level is about 1245 nM and a level of the TBA greater than the cut-off level is indicative the donor lung is not suitable for transplantation and a level of TBA equal to or less than the cut-off level is indicative the donor lung is suitable for transplantation, wherein the cut-off level is calculated by instilling and suctioning 20 ml of normal saline. The person skilled in art would understand the cut-off levels depend on the volume of normal saline being used. For instance, if 40 mL of normal saline is used, the cut-off level would be half at about 622.5 nM, since the volume has doubled and so the concentration is halved.

In an embodiment, the one or more inflammation markers are measured and are selected from interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10).

It will be understood that different combinations of biomarkers including bile acid and inflammation marker can be used in any of the methods described herein.

In an embodiment the one or more inflammation markers are selected from interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10).

In an embodiment, the one or more inflammation markers comprises or is IL-6.

In an embodiment, the one or more inflammation markers comprises or is IL-8.

In an embodiment, the one or more inflammation markers comprises or is IL-6 and IL-8.

In an embodiment, the donor lung being assessed is undergoing EVLP and if the donor lung is identified as not suitable for transplant, the donor lung is rejected for transplant or remain in EVLP for further assessment.

In an embodiment, the method is carried out at point-of-care.

A further aspect relates to a method of detecting of bile acid in the bronchial wash sample to predict GERD and optionally help circumvent issues related thereto, including for example chronic lung inflammation and chronic lung allograft dysfunction.

Accordingly, another aspect of the present disclosure is a method of assessing the likelihood of developing gastroesophageal reflux disease (GERD) in a donor lung recipient, the method comprising:
a) obtaining a bronchial wash sample from the donor lung recipient;
b) measuring a level of a bile acid in the bronchial wash sample; and
c) comparing the level of the bile acid with a bile acid cut-off level, and
d) identifying the donor lung recipient as likely to develop GERD when the level of the bile acid is greater than the bile acid cut-off level, or unlikely to develop GERD when the bile acid level is equal to or less than the bile acid cut-off level, optionally wherein the bile acid cut-off level is determined by calculating in a population of donor lungs an average bile acid cut-off level above which at least 90% of the donor lungs were identified as having developed GERD.

In an embodiment, when the level of the bile acid is greater than the bile acid cut-off level, the method described herein further comprises performing EVLP to confirm lung function prior to transplantation.

In an embodiment, when the level of the bile acid is greater than the bile acid cut-off level, the method described herein further comprises treating the lungs for aspiration damage. In an embodiment, the method comprises treating the lungs with surfactant.

In an embodiment, when the level of the bile acid is greater than the bile acid cut-off level, the method described herein further comprises rejecting the lung for transplantation.

In an embodiment, the bronchial wash sample is a BAL sample. In an embodiment, the bronchial wash sample is a LABW sample.

In an embodiment, the bronchial wash sample is a BAL sample.

In an embodiment, the bronchial wash sample is a LABW sample.

In another embodiment, the bile acid is selected from one or more of taurocholic acid (TCA), glycocholic acid (GCA), cholic acid (CA), and combinations thereof.

In an embodiment, the bile acid is total bile acid (TBA).

The levels of the biomarkers can be detected using a number of methods known in the art. In an embodiment, the bile acid and optionally the one or more inflammation markers are detected or determined by Luminex® based assays, Western blots, immunoassay e.g. ELISA, immunofluorescence, radioimmunoassay, dot blotting, FACS, protein microarray, immunoprecipitation followed by SDS-PAGE, immunocytochemistry, simple Plex assay, multiplex assay, mass spectrometry, electrochemical assay, enzymatic assay or colorimetric assay.

Enzymatic cycling reaction is a routine method to clinically detect TBA. This method can quantify TBA indirectly by UV or fluorescence detection of NADH produced by NAD⁺ in the oxidation reaction of BAs. The use of spectrophotometric enzymatic cycling assay at the point-of-care is known in the art. In an embodiment, the level of the bile acid is detected using an enzymatic assay such as a spectrophotometric enzymatic cycling assay. In an embodiment, the spectrophotometric enzymatic cycling assay is described in Example 1 and Example 3. Enzymatic method detection can also be coupled with electrochemical detection, as bile acids can be quantified on the basis of electrical signals generated through oxidation of the product NADH on the surface of screen-printed carbon electrodes. Electrochemical measurements can be performed on a system such as a Bioanalytical Systems (BASi) epsilon potentiostat with a three-electrode system featuring an Ag/AgCl reference electrode (BASi), a platinum wire auxiliary electrode, and a biosensing electrode serving as the working electrode. Electrodes can be incubated in a solution such as a solution of NAD+/NADH, 3α-HSD and lung wash sample, then scanned using differential pulse voltammetry (DPV). In an embodiment, the level of the bile acid is detected using an electrochemical assay.

In an embodiment, the level of the bile acid is detected using an immunoassay, optionally an ELISA.

In an embodiment, the level of the bile acid is detected using mass spectrometry.

### III. Assays, Kits and Devices

An aspect of the disclosure also includes assays, devices and kits for the detection of the biomarkers of the disclosure that are used to measure the biomarker levels.

In an embodiment, the assay, devices and kits are suitable for use at point-of-care.

An aspect herein disclosed is a kit comprising at least one detection agent specific for a biomarker selected from bile acid, optionally total bile acid, taurocholic acid (TCA), glycocholic acid (GCA) or cholic acid (CA), and optionally an inflammation marker, optionally interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10), the kit optionally further comprising one or more of a 96-well plate, standards, assay buffer, wash buffer, sample diluent, standard diluent, detection antibody diluent, streptavidin-PE, a filter plate and sealing tape, optionally for performing the methods disclosed herein.

In an embodiment, the bile acid is total bile acid (TBA) or a component thereof selected from taurocholic acid (TCA), glycocholic acid (GCA), cholic acid (CA), and combinations thereof.

In an embodiment, the one or more inflammation markers are selected from interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10).

In an embodiment the kit comprises an immunoassay for one or more of biomarkers of the disclosure. Each kit comprises at least one detection antibody specific for a biomarker of the disclosure. For example, the antibody can be in the form of antibody coupled beads such as antibody coupled magnetic beads, or labelled antibodies, optionally comprised in a cartridge. In an embodiment, the kit further comprises one or more of a 96-well plate, a cartridge comprising one or more antibodies, standards, assay buffer, wash buffer, sample diluent, standard diluent, detection antibody diluent, streptavidin-PE, a filter plate and sealing tape. In an embodiment the kit comprises detection antibodies or assays for detecting two or more biomarkers of the disclosure e.g. two or more of bile acid (e.g. TCA, GCA, CA), IL-8 and IL-6. In an embodiment, the kit comprises detection antibodies or assays for detecting TCA and GCA. In an embodiment, the kit comprises detection antibodies or assays for detecting bile acid TCA and CA. In an embodiment, the kit comprises detection antibodies or assays for detecting bile acid GCA and CA. In an embodiment, the kit comprises detection antibodies or assays for detecting bile acid (e.g. TCA, GCA, CA), IL-6 and IL-8.

In an embodiment, the kits are for use for a method described herein.

Another aspect is a point-of-care (POC) device for detecting the presence of bile acid in a sample. The device comprises:
a housing,
a porous medium comprised in the housing, the porous medium comprising at a first end a sample portion and at a second end a testing portion,
a channel defined between the sample portion and the testing portion, the channel defining a flow path along which the sample passes and is drawn from the sample portion to the testing portion by capillary action, and
one or more reagents or detection agents for bile acid, optionally in fluid communication with the testing portion or immobilized on the testing portion, the one or more reagents or detection agents being capable of reacting with the bile acid to generate a detection signal, optionally a visible color change or a fluorescent signal.

In an embodiment, the bile acid is selected from total bile acid (TBA), or a component thereof selected from taurocholic acid (TCA), glycocholic acid (GCA), cholic acid (CA), and combinations thereof.

In an embodiment, the device further comprises one or more reagents detection agents for inflammation markers, optionally selected from interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10).

In an embodiment, a sample delivered into or on the sample portion migrates to the testing portion via capillary action.

In an embodiment, the sample is a bronchial wash sample, optionally a bronchoalveolar lavage (BAL) sample or a large airway bronchial wash (LABW) sample.

In an embodiment, the devices described herein can also be described as lateral flow devices. Herein, the term “lateral flow device” generally refers to a device that includes one or more fluid channels, chambers or conduits that spontaneously drive a fluid across the device (e.g. by capillary force). Lateral flow device is well known in the art and it can be in a variety of formats such as sandwich format, competitive format, and multiplex format. Lateral flow device can also be used with a variety of affinity reagents, such as antibodies and aptamers (see e.g., Sajid M, Kawde A and Daud M (2015) Design, Formats and Applications of Lateral Flow Assays, J Saudi Chem Soc., 19, 689-705, and Bahadir EB & Sezgintürk MK (2016) Lateral flow assays: Principles, designs and labels. Trends in Analytical Chemistry, 82, 286-306, the contents of which are incorporated by reference herein in their entireties). It is known in the art that sensitivity of a lateral flow device is affected by various factors such as pore size of the nitrocellulose membrane and flow rate, and experimental conditions can be optimized for lateral flow device performance (See Bahadir, E. B., & Sezgintürk, M. K. (2016)). In an embodiment, the POC device comprises a lateral flow device wherein the lateral flow device has been optimized such that the level of the bile acid or inflammation marker is not detectable in samples from suitable graft, but detectable in samples from poor graft. In some embodiments, the level of the bile acid or inflammation marker is not detectable in sample from suitable graft, but detectable in samples from poor graft. It can be understood that a lateral flow device for detecting or measuring bile acid or inflammation markers disclosed herein can be optimized based on those factors affecting sensitivity. The skilled person can also readily configure a lateral flow device that provides control signals for ease of use or to increase the accuracy of the device. In an embodiment, the lateral flow device comprises a control line for checking device function. In an embodiment, the lateral flow device comprises a control link for checking assay function. v

The lateral flow device described herein is intended for rapid detection of the presence of an analyte in a test sample without the need for costly or sophisticated equipment. This device is useful in POC applications, as well as in laboratory testing or medical diagnostics. There are a number of configurations for a lateral flow device known to the person skilled in the art. For example, the lateral flow device described herein can comprise a sensor portion having an immobilized and stabilized sensor, and a testing portion having stabilized reporting solution. In an embodiment, the lateral flow device comprises a buffer portion for applying a running buffer, the buffer portion being connected through a flow channel to ii) a sensor portion for applying a sample, the sensor portion being connected through a flow channel to iii) a testing portion for indicating the presence, absence, or a range of levels of an analyte. In an embodiment, the running buffer comprises one or more reagents or detection agents. In an embodiment, the buffer portion is immobilized to a solid support. In an embodiment, the sensor portion is immobilized to a solid support. In an embodiment, the testing portion is immobilized to a solid support. In an embodiment, the testing portion comprises a plurality of testing zones. In an embodiment, the buffer portion, the sensor portion, and the testing portion are immobilized on a solid support. In an embodiment, the buffer portion comprises one or more reagents or detection agents for an analyte. In an embodiment, the sensor portion comprises one or more reagents or detection agents for an analyte. In an embodiment, the testing portion comprises one or more reagents or detection agents for an analyte. In an embodiment, the buffer portion comprises one or more analyte-specific antibodies. In an embodiment, the sensor portion comprises one or more analyte-specific antibodies. In an embodiment, the testing portion comprises one or more analyte-specific antibodies. In an embodiment, the analyte is one or more bile acids. In an embodiment, the analyte is one or more inflammation markers. In an embodiment, the one or more analyte-specific antibodies are specific for one or more bile acids. In an embodiment, the one or more analyte-specific antibodies are specific for one or more inflammation markers. In an embodiment, the one or more analyte-specific antibodies are coupled to a detectable label. In an embodiment, the one or more reagents or detection agents are capable of reacting with the analyte and/or the one or more analyte-specific antibodies to generate a detection signal. In an embodiment, the detection signal is a visible color change. In an embodiment, the detection signal is a fluorescent signal. In an embodiment, the color change or fluorescent signal is proportionate to the amount of one or more bile acids. In an embodiment, the color change or fluorescent signal is titrated with the amount of one or more bile acids. In an embodiment, the signal, optionally a fluorescent signal, instill is detected by a reader. The skilled person can recognize suitable fluorescent label for the generation of a fluorescent signal, and suitable reader for detecting the fluorescent signal.

In another embodiment, the lateral flow device comprises a sample portion and a testing portion. In an embodiment, the sample portion and the testing portion are connected through a flow channel. In an embodiment, the sample portion is for applying a running buffer and an analyte. In an embodiment, the sample portion is for applying a mixture comprising a running buffer and an analyte. In an embodiment, the lateral flow device comprises a sample portion for applying a mixture of a running buffer and an analyte, the sample portion being connected through a flow channel to a testing portion for indicating the presence, absence, or a range of levels of an analyte. In an embodiment, the sample portion is immobilized to a solid support. In an embodiment, the testing portion is immobilized to a solid support. In an embodiment, the sample portion and the testing portion are immobilized to a solid support. In an embodiment, the testing portion comprises a plurality of testing zones. In an embodiment, the sample portion comprises one or more reagents or detect agents for an analyte. In an embodiment, the testing portion comprises one or more reagents or detect agents for an analyte. In an embodiment, the sample portion comprises one or more analyte-specific antibodies. In an embodiment, the testing portion comprises one or more analyte-specific antibodies. In an embodiment, the sample portion and the testing portion each comprises one or more analyte-specific antibodies. In an embodiment, the analyte is one or more bile acids. In an embodiment, the analyte is one or more inflammation markers. In an embodiment, the one or more analyte-specific antibodies are specific for one or more bile acids. In an embodiment, the one or more analyte-specific antibodies are specific for one or more inflammation markers. In an embodiment, the one or more analyte-specific antibodies are coupled to a detectable label. In an embodiment, the one or more reagents or detection agents are capable of reacting with the analyte and/or the one or more analyte-specific antibodies to generate a detection signal. In an embodiment, the detection signal is a visible color change. In an embodiment, the detection signal is a fluorescent signal. In an embodiment, the color change or fluorescent signal is proportionate to the amount of one or more bile acids. In an embodiment, the fluorescent signal is detected by a reader. The skilled person can recognize suitable fluorescent label for the generation of a fluorescent signal, and suitable reader for detecting the fluorescent signal.

The lateral flow device described herein can involve the use of a conjugate comprising an antibody coupled to a detectable label, such as a colored particle such as a metal sol or colloid. In an embodiment, the detectable label is an enzymatic moiety. In an embodiment, the enzymatic moiety comprises urease, alkaline phosphatase, horseradish peroxidase, glucose oxidase, or ß-galactosidase. In an embodiment, the enzymatic moiety is a pH changing enzyme. In an embodiment, the pH changing enzyme comprises urease, and the substrate comprises urea, and the color changing dye comprises bromothymol blue, phenol red, neutral red, cresol red, m-cresol purple, or o-cresolphthalein complexone. In an embodiment, the enzymatic moiety comprises horseradish peroxidase, and the substrate comprises 2,2′-Azinobis [3-ethylbenzothiazoline-6-sulfonic acid]-diammonium salt (ABTS), 3-amino-9-ethylcarbazole (AEC), 3,3-diaminobenzidine (DAB), 3,3′,5,5′-Tetramethylbenzidine (TMB), or Amplex Red. In an embodiment, urease catalyzes the conversion of urea to ammonia, which increases the pH of the solution, which can then be detected by a change in color of the color changing dye. In an embodiment, the metal sol or colloid is gold.

The conjugate can be in a form suitable for, e.g. sandwich or competitive format. When the lateral flow device relates to the sandwich format, it can involve a first antibody that is capable of binding to a first epitope on the analyte (e.g. bile acid), and by which the firs antibody is immobilized at the testing zone of the testing portion of the lateral flow device. Under this format, there is also a conjugate comprising a detectable label and a second antibody that is capable of binding to a second epitope on the analyte, and the binding of the conjugate to the analyte would form a complex. The formation of this complex is detected at the testing zone, where the complex reacts with the immobilized first antibody to form a “sandwich” of the first antibody, analyte, and conjugate (i.e. the second antibody coupled with a detectable label). This sandwich comprising the analyte is detected by visual observation of color development, as the sandwich is progressively produced at the testing zones where the conjugate-analyte complex is captured by the first antibody. As more conjugate is immobilized for the formation of the sandwich, the detectable labels aggregate at the testing zones and become visible because they are colored, or through an enzymatic reaction, indicating the presence of the analyte in the sample. The skilled person can also readily construct a device that produces a signal when the amount of the analyte is above a cut-off threshold described herein.

When the lateral flow device relates to the competitive format, it can involve a conjugate comprising an authentic sample of the analyte and a detectable label. The conjugate would be in competition with the analyte for binding to an analyte-specific antibody, which is immobilized at the testing zone. As the sample and the conjugate flow through the device and arrive at the testing zones, any analyte in the sample would compete with the conjugate for sites of attachment to the analyte-specific antibody. If no analyte is in the sample, the detectable labels aggregate at the testing zone, and the development of color, for instance because the detectable label is colored or through an enzymatic reaction, indicates the absence of detectable levels of the analyte in the sample. If the analyte is in the sample, the amount of conjugate which binds at the testing zone is reduced (i.e. competed out by the analyte), and no color, or a lighter color, is observed. The skilled person can also readily construct a device that produces a signal when the amount of the analyte is above a cut-off threshold described herein.

A number of materials are useful for making the lateral flow device described herein. The lateral flow device can have separated zones and flow channels. Such zones and flow channels can be created by wax on a nitrocellulose paper backed with a plastic sheet, i.e. wax acts as a uniform hydrophobic barrier for which the running buffer does not penetrate and the nitrocellulose paper acts to allow lateral flow of the running buffer. Methods for creating a hydrophobic barrier on a support layer are known to the person skilled in the art. The skilled person also recognizes that many alternatives to nitrocellulose paper are possible, for example, any material that allows flow could work, such as cellulose, or any other surface that supports capillary flow. Accordingly, in an embodiment, the lateral flow device comprises nitrocellulose paper, cellulose, or any surface that supports capillary flow. In an embodiment, the lateral flow device comprises nitrocellulose paper. In an embodiment, the lateral flow device comprises a polymer support layer. In an embodiment, the polymer support layer comprises a plastic sheet. In an embodiment, the lateral flow device comprises a hydrophobic material. In an embodiment, the hydrophobic material comprises wax. In an embodiment, the lateral flow device was printed by a hydrophobic material. In an embodiment, the lateral flow device was printed by wax.

In an embodiment, the lateral flow device described herein is for use in identifying the donor lung as likely to have had significant aspiration when the level of bile acid provides a different signal output as compared to the cut-off level, or unlikely to have had significant aspiration when the level of the bile acid provides a similar signal output as compared to a cut-off level, or identifying the donor lung as likely to have had significant aspiration when the level of the level of bile acid provides a similar signal output as compared to a cut-off level or device, or unlikely to have had significant aspiration when the level of the bile acid provides a different output as compared to a cut-off level, optionally, transplanting the donor lung in a recipient if the donor lung is unlikely to have had significant aspiration.

In an embodiment, the device is for use for a method described herein.

The above disclosure generally describes the present disclosure. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1—Bile Acids and Inflammation Markers in Assessing the Suitability of Donor Lungs for Transplantation

### Materials and Methods

### Study Design

A retrospective cohort study including all consecutive lung transplant donors at the Toronto Lung Transplant Program from 2012 to 2018 with available LABW was conducted.

### LABW Sample Collection

As per the International Society for Heart and Lung Transplantation Consensus Statement for the Standardization of Bronchoalveolar Lavage in Lung Transplantation, LABW was collected by instilling and suctioning 20 ml of normal saline through a flexible bronchoscope in the mainstem bronchus at time of organ retrieval in the donor operating room (Martinu et al. J Heart Lung Transplant, “International Society for Heart and Lung Transplantation consensus statement for the standardization of bronchoalveolar lavage in lung transplantation”, 2020 November; 39(11): 1171-1190; PMID: 32773322). Samples were then centrifuged for 20 min at 3184 g at 4° C., supernatants were separated and frozen at −80° C.

### Ex Vivo Lung Perfusion Protocol

Donor lungs were placed on the EVLP circuit, perfused, and ventilated for up to 6 hours. Perfusate was collected at the arterial and venous ends every hour and assessed immediately for partial pressure of oxygen (pO₂) at FiO₂ of 100%, partial pressure of carbon dioxide (pCO₂) at FiO₂ of 100%, markers of acid/base chemistry (pH, glucose, lactate, base excess, and HCO₃⁻) using a blood gas monitoring system (Siemens, Erlangen, Germany), and electrolytes (Na⁺, K⁺, Ca²⁺, and Cl⁻). Difference in the partial pressure of oxygen and carbon dioxide between the arterial and venous ends were calculated at each hour and used for later analysis. A sample of EVLP perfusate was also snap frozen in liquid nitrogen at each hour and stored for later analysis. Pulmonary physiologic parameters including pulmonary vascular resistance (PVR) and lung compliance (dynamic and static) were measured hourly.

### TBA and Protein Measurements

All samples were thawed at 4° C. overnight. TBA was measured in donor LABW using a spectrophotometric enzymatic cycling assay kit (BQ Kits, Inc., San Diego, CA) using the Cobas diagnostic platform (Roche, Rotkreuz, Switzerland). Inflammatory mediators (GM-CSF, IL-10, IL-1ß, IL-6, IL-8, sTNFR1, and sTREM1) were measured using the ELISA-based ELLA platform (Protein Simple, San Jose, CA) as per manufacturer's instructions. Protein concentrations are reported as adjusted and normalized values in pg/mL/L after correcting for lung size using Total Lung Capacity (in L) and effects of perfusate volume exchanges during EVLP.

### Post-Transplant Outcome Definitions

Follow-up data on transplant recipients was obtained from electronic medical records and computerized databases.

Acute Rejection: Transbronchial biopsies were clinically graded for acute vascular (A-grade) and airway inflammation (B-grade) rejection as per the International Society of Heart and Lung Transplantation consensus guidelines (Stewart et al. JHLT 2007). A and B scores were calculated by averaging A and B grades within the 7 months post-transplant period, respectively. Recipients with biopsies failing to meet minimum standard (AX and/or BX) and those with no biopsy within the 7 months post-transplant period were excluded from the analysis.

Infection Score is a measure of infection and is defined as the number of BAL samples positive for infectious pathogens divided by the total number of BAL samples available over a given time period

PGD is defined by the presence of pulmonary edema on chest X-ray and the PaO2/FiO2 ratio according to the 2016 International Society for Heart and Lung Transplantation Primary Graft Dysfunction Definition (Snell et al. JHLT 2017)

Chronic lung allograft dysfunction (CLAD) is defined as a sustained and irreversible decline in forced expiratory volume in one second (FEV1)≤80% (based on two FEV1 values separated by at least three weeks) of the post-transplant baseline (defined as the average of the two highest post-transplant FEV1 values at least three weeks apart in the absence of other confounding etiologies). Date of CLAD onset was defined as the date of the first FEV1≤80% of baseline in accordance with consensus guidelines from the International Society for Heart and Lung Transplantation (Verleden et al. JHLT 2019).

### Statistical Analysis

SPSS version 27 (Microsoft, IBM Corporation, Armonk, NY) was used for all statistical analysis. Data was visualized using the packages Seaborn (Waskom JOSS 2021) and kaplanmeier (https://github.com/erdogant/kaplanmeier) in Python. To compare between different groups of lung donors and transplant recipients in the primary cross-sectional analysis, Wilcoxon-Mann-Whitney and Fischer's exact tests were used for continuous and categorical variables, respectively. Correlations between donor LABW TBA concentrations and biomarkers in EVLP perfusate were evaluated using Spearman's correlation.

The predictive ability of TBA to identify donor organs suitable for direct transplant versus unsuitable for transplantation was assessed using the area under receiver operating characteristic curve (AUROC), using a null hypothesis that the AUROC was 50%. A 90% specificity threshold was used to determine the TBA cut-off to identify donor lungs with significant aspiration. Multivariable Cox proportional hazard models were used to assess the association between donor lungs with high TBA concentrations and recipient time to extubation, time to ICU discharge, CLAD, and death.

### Results

### Baseline Clinical Characteristics

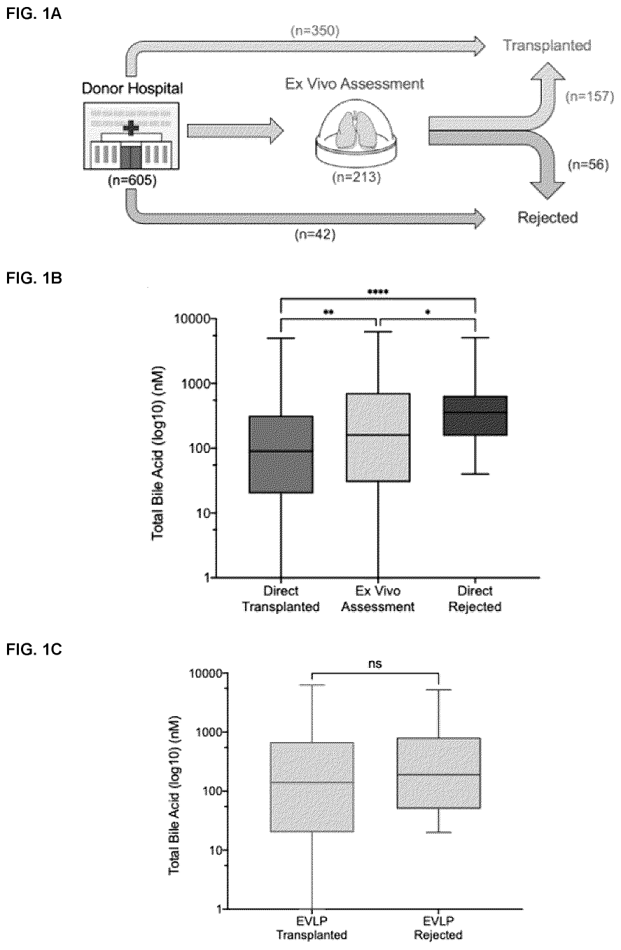

A total of 605 consecutive lung donors at the Toronto Lung Transplant Program from 2012-2018 with available LABW were included in this retrospective study. From this group, 350 organs were accepted for direct transplant, 42 organs were declined for transplant, and 213 were further assessed on EVLP. Of the EVLP group, 157 donor lungs were accepted for transplant and 56 donor lungs were declined (FIG. 1A). As previously reported, donor age, sex, type of death, and BMI were significantly different between lungs directly transplanted vs those first assessed on EVLP before transplant (Divithotawela et al. JAMA Surg 2019). There were no significant differences in recipient characteristics between those who received lungs that were directly transplanted vs those that were first assessed on EVLP, as shown in Table 1 below.

TABLE 1
Demographics of study participants

| | Direct Lung | | EVLP Lung | | |
|---|---|---|---|---|---|
| | Transplant | | Transplant | | |
| | (n = 350) | | (n = 157) | | p-value |
| Donor age | | | | | |
| Median (IQR) | 52 | (36-63) | 46 | (31-55) | 0.002 |
| Male, n (%) | 203 | (58) | 111 | (71) | 0.006 |
| BMI (kg/m²) | | | | | |
| Median (IQR) | 26 | (23-30) | 27 | (24-32) | 0.02 |
| Donor Type, n (%) | | | | | <0.001 |
| DBD | 319 | (91) | 180 | (51) | |
| DCD | 31 | (9) | 77 | (49) | |
| Ever cigarette use, n (%) | 172 | (49) | 84 | (54) | 0.36 |
| Recipient age at transplant | | | | | |
| Median (IQR) | 56 | (44-62) | 58 | (45-64) | 0.62 |
| Male, n (%) | 188 | (54) | 91 | (57) | 0.37 |
| BMI (kg/m²) | | | | | |
| Median (IQR) | 23 | (20-27) | 24 | (21-28) | 0.59 |
| Single lung transplant, n (%) | 23 | (7) | 13 | (8) | 0.49 |
| Heart-lung transplant, n (%) | 4 | (1) | 0 | (0) | 0.17 |
| Native lung disease, n (%) | | | | | 0.36 |
| Chronic obstructive | 70 | (20) | 31 | (20) | |
| pulmonary disease | | | | | |
| Cystic Fibrosis | 56 | (16) | 16 | (10) | |
| Pulmonary Fibrosis | 142 | (41) | 71 | (45) | |
| Other | 82 | (23) | 39 | (24) | |
| CMV mismatch | 82 | (23) | 36 | (23) | 0.90 |
| (D+/R−), n (%) | | | | | |
| Waitlist time in months, | 3 | (1-6) | 3 | (1-8) | 0.51 |
| median (IQR) | | | | | |
| Status at time of transplant, | | | | | 0.78 |
| n (%) | | | | | |
| 1 | 87 | (25) | 44 | (28) | |
| 2 | 136 | (39) | 59 | (38) | |
| 3 | 124 | (35) | 54 | (34) | |

Donor TBA was Associated with Suitability of Lungs for Transplant

TBA measured in donor LABW was compared between lungs suitable for direct transplantation, unsuitable for transplant, and those requiring further assessment on EVLP. Donor lungs unsuitable for transplantation had higher levels of TBA than those suitable for direct transplantation (p<0.001) and those requiring further assessment on EVLP (p<0.01). Donor lungs that were unsuitable for transplant had higher levels of TBA than lungs that required assessment on EVLP (p=0.04) (FIG. 1B). Among donor lungs that were assessed on EVLP, there was no difference in TBA concentration between those that were accepted vs declined for transplant (p=0.30) (FIG. 1C).

Donor TBA was Associated with Calcium, Acidemia, and Inflammatory Mediators in EVLP Perfusate

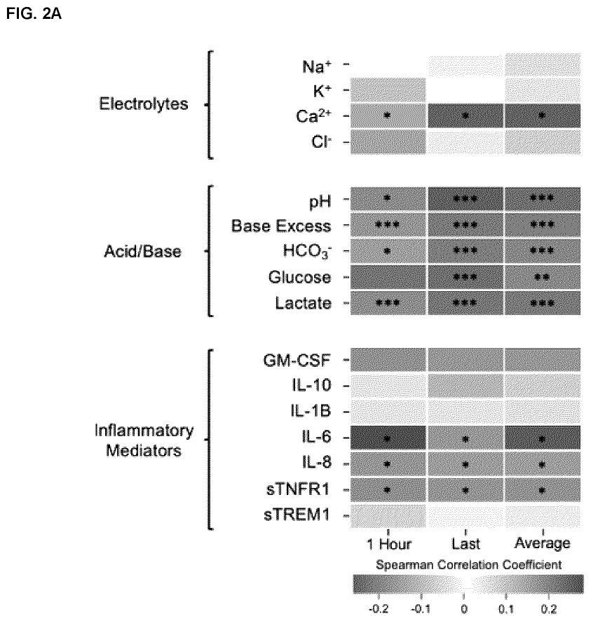

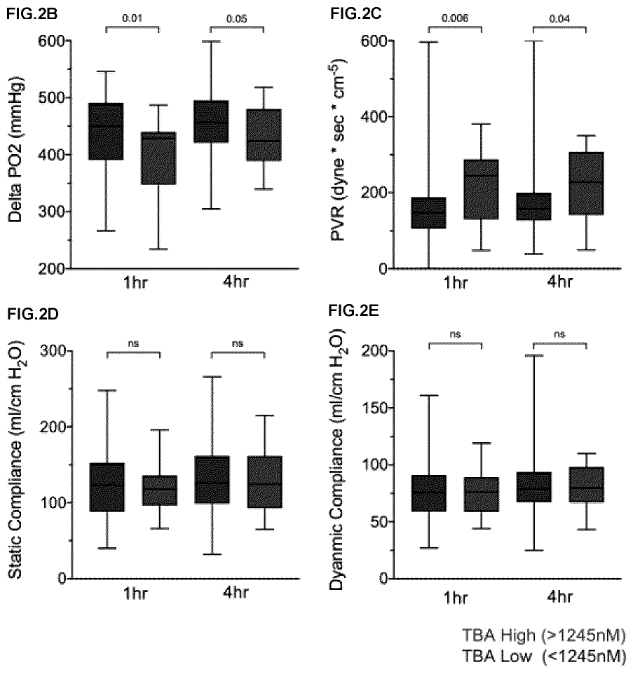

Electrolytes, markers of acid/base chemistry, and inflammatory mediator proteins were measured every hour in EVLP perfusate and compared to donor LABW TBA concentrations. FIG. 2A summarizes the Spearman correlations between donor TBA and markers in EVLP perfusate. Of note, average Ca²⁺ (r=0.25; p<0.001), pH (r=−0.23; p<0.001), base excess (r=−0.21; p<0.001), HCO₃⁻ (r=−0.19; p<0.001), glucose (r=−0.19; p=0.01), lactate (r=0.22; p<0.001), IL-6 (r=0.26; p=0.01), IL-8 (r=0.16; p=0.03), and sTNFR1 (r=0.18; p=0.03) in EVLP perfusate were associated with donor LABW TBA. Except for glucose, these markers were also associated with donor TBA after one hour on EVLP and at the last hour before taking lungs off EVLP. Glucose was not associated with donor TBA after one hour of EVLP but was associated with donor TBA at the last hour of EVLP.

Identifying Donors with Significant Aspiration Using TBA Concentration

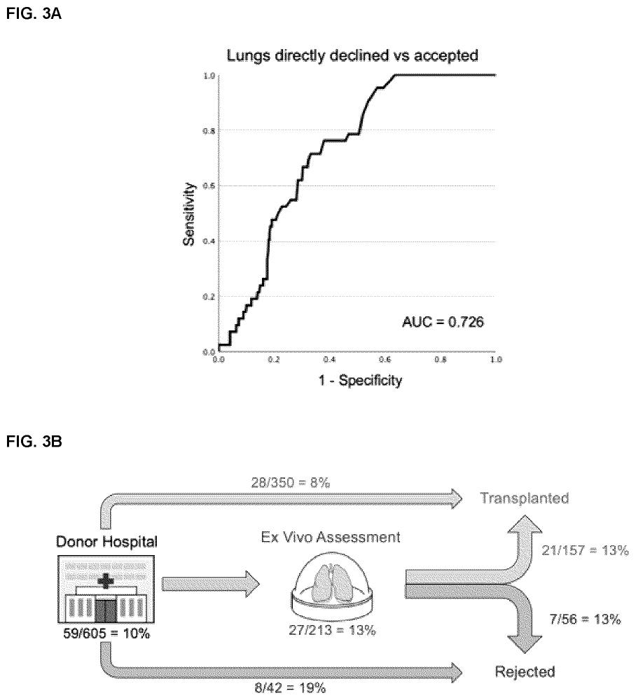

As aspiration is a contraindication for accepting donor lungs and bile acid in the airways is a specific marker of aspiration, donors with significant aspiration were identified using their LABW TBA concentration. Area under the receiver operator characteristic curve was 72.6% when using donor TBA to predict lungs suitable for direct transplantation vs those unsuitable for transplant (FIG. 3A). TBA cut-off of 1245 nM was able to identify donor lungs unsuitable for transplant with 91% specificity.

In the single-centre retrospective cohort, the prevalence of TBA>1245 nM was: 10% among all lung donors (n=59), 19% among lungs unsuitable for transplant (n=8), 8% among lungs directly transplanted (n=28) and 13% among lungs requiring further assessment on EVLP (n=27). The prevalence of TBA>1245 nM was 13% among both lungs transplanted (n=21) and declined after EVLP (n=7) (FIG. 3B). Demographics of donors whose lungs were used for transplant with high vs low TBA were not different with respect to age, sex, BMI, donor type (donor before brain vs cardiac death), nor ever cigarette use. Individuals who received donor lungs with high vs low TBA concentration did not differ in age at transplant, sex, BMI, type of transplant, native lung disease, CMV mismatch, and status at time of transplant.

Donor TBA Status was Associated with PCO₂ and Pulmonary Vascular Resistance During EVLP

The association between donor TBA status and measurements of lung physiology at every hour on EVLP was investigated (Mann-Whitney test). Donor lungs with high TBA had a lower delta PO₂ after one hour (p=0.01) and four hours (p=0.05) on EVLP (FIG. 2B). High TBA status was also associated with higher pulmonary vascular resistance at one hour (p=0.006) and four hours (p=0.04) on EVLP (FIG. 2C). In contrast, neither static nor dynamic pulmonary compliance were associated with donor TBA status (FIG. 2D-2E).

### Donor TBA Status was Predictive of Recipient Outcomes

Donor TBA status was compared to recipient bronchoscopy-based characteristics in the 7 months post-transplant period: A score, B score, and infection score. High TBA status was associated with higher B score, however there was no difference in A score nor infection score between the two groups. Results are shown in Table 2 below.

TABLE 2
Associations between donor TBA status and recipient outcome

| Bronchoscopy based | | | |
|---|---|---|---|
| characteristics | TBA <1245 nM | TBA >1245 nM | p-value |
| A Score^{a} | 0.26 (0-1.50) | 0.26 (0-0.75) | 0.74 |
| B Score^{a} | 0.03 (0-1.00) | 0.08 (0-1.00) | 0.04 |
| Infection Score^{a} | 0.36 (0-1.00) | 0.36 (0-1.00) | 0.96 |

| Outcome^{c} | Hazard Ratio (95% CI) | p-value |
|---|---|---|
| Time to extubation | 0.39 (0.29-0.55) | <0.001 |
| ICU length of stay | 0.42 (0.28-0.63) | <0.001 |
| CLAD | 1.64 (1.03-2.63) | 0.04 |
| Death or retransplant | 1.43 (0.96-2.14) | 0.12 |

Abbreviations: CLAD—chronic lung allograft dysfunction
^{a}Data represents mean (range) with Mann-Whitney Test to assess statistical significance.
^{b}Data represents n (%) with Chi-squared Test to assess statistical significance.
^{c}Independent variable is donor TBA status high (TBA >1245 nM) vs low (TBA <1245 nM).

Results from multivariable cox proportional hazards models adjusting for donor: age, sex, and type of death as well as recipient: age, sex, CMV mismatch, native lung disease, and status at time of transplant. Bolded values represent statistical significance.

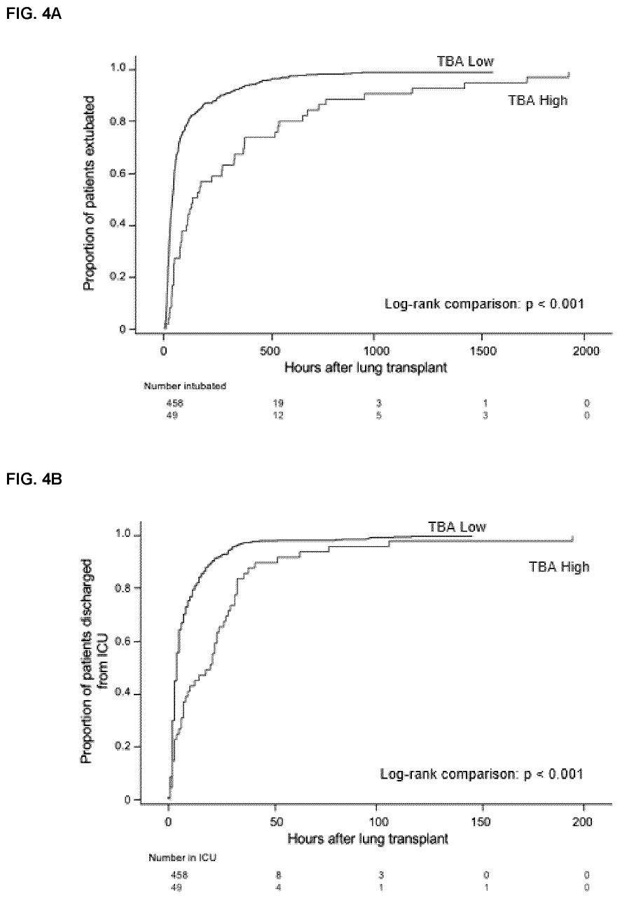

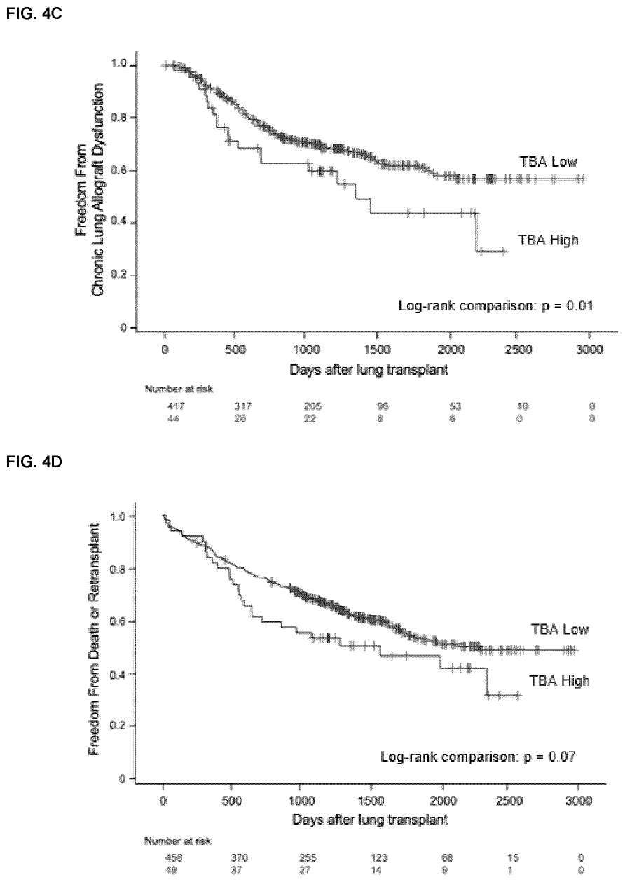

The ability of donor TBA status to predict recipient outcomes was assessed using a multivariable Cox proportional hazards model adjusting for donor: age, sex, and type of death as well as recipient: age, sex, CMV mismatch, native lung disease, and status at time of transplant. In this multivariable analysis, as shown in Table 2 and FIG. 4, high donor TBA status was associated with longer time to extubation (FIG. 4A), longer time to ICU discharge (FIG. 4B), and decreased time to CLAD (FIG. 4C). However, high donor TBA status was not associated with time to death or retransplant (FIG. 4D).

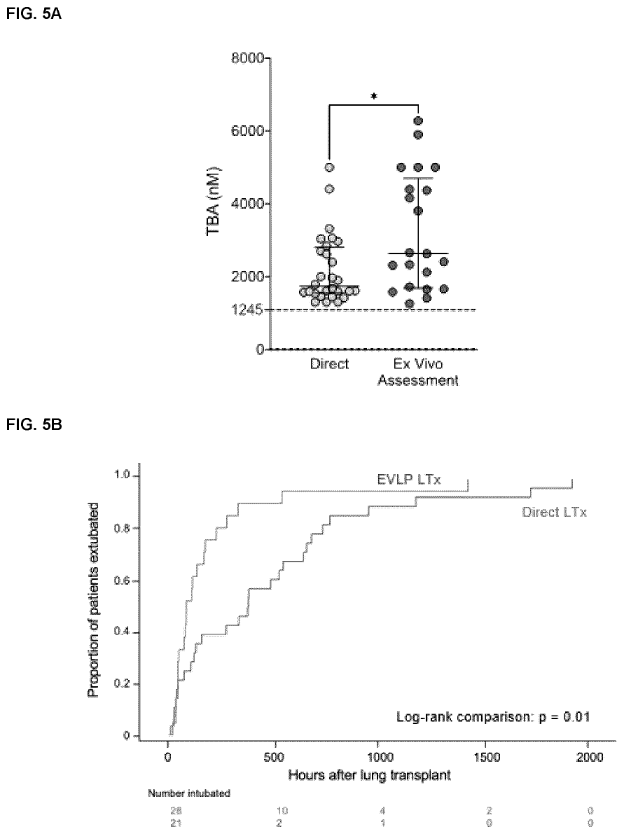

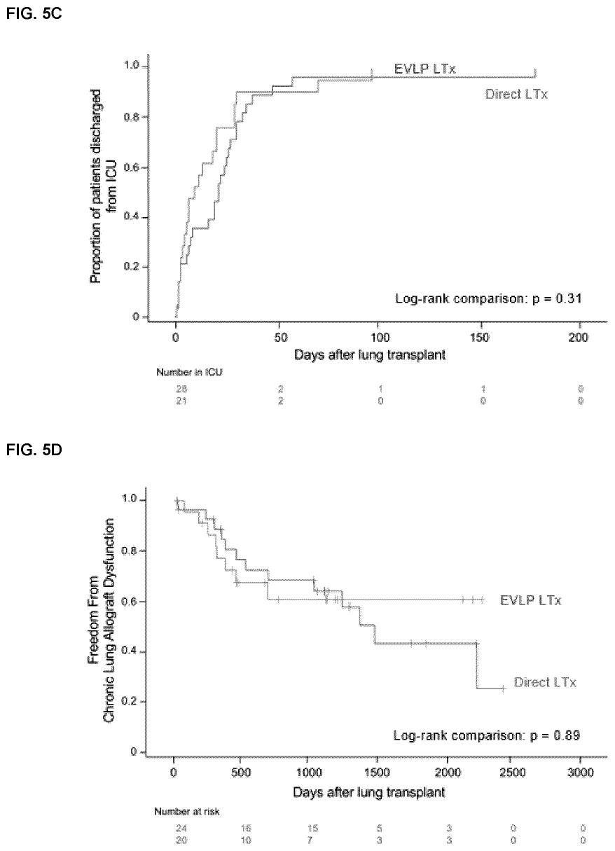

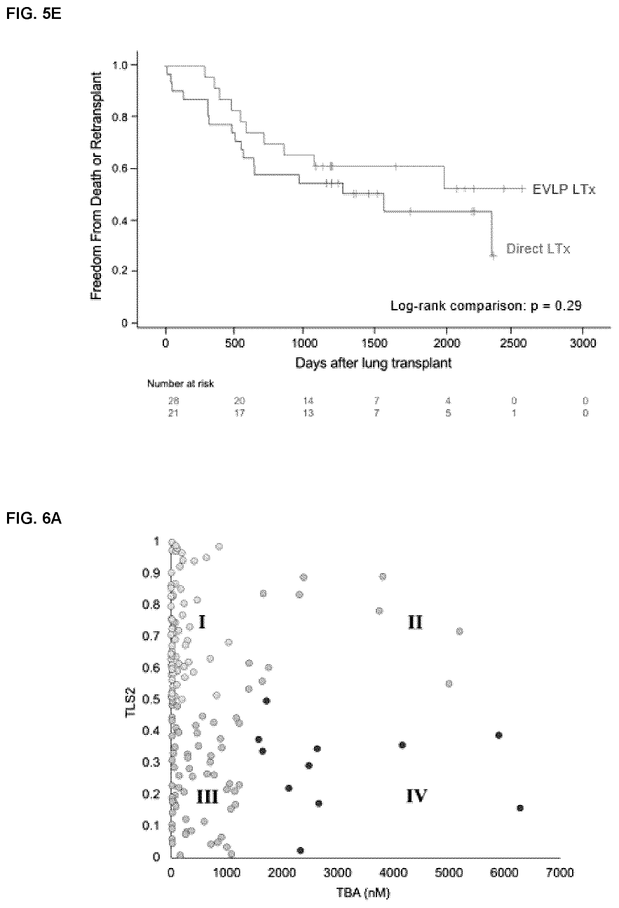

EVLP allows for potentially damaged donor lungs to be further assessed before deciding whether the donor organ is suitable for transplant. Differences in recipient outcomes between transplanted donor lungs with high TBA that were either directly transplanted (n=28) or assessed on EVLP before transplant (n=21) were assessed. Median TBA concentration was higher in EVLP lungs with TBA>1245 nM than direct to transplant lungs with TBA>1245 nM (p=0.03) (Mann-Whitney test) (FIG. 5A). Recipients with directly transplanted high TBA lungs had a longer time to extubation compared those who received high TBA lungs that were first assessed on EVLP (p=0.01) (Log-rank comparison) (FIG. 5B). Time to ICU discharge (p=0.31) (FIG. 5C), time to CLAD (p=0.89) (FIG. 5C), and time to death or retransplant (p=0.29) (FIG. 5D) were not different between these two groups.

### Combining TBA and Toronto Lung Score Predicts Recipient Outcome

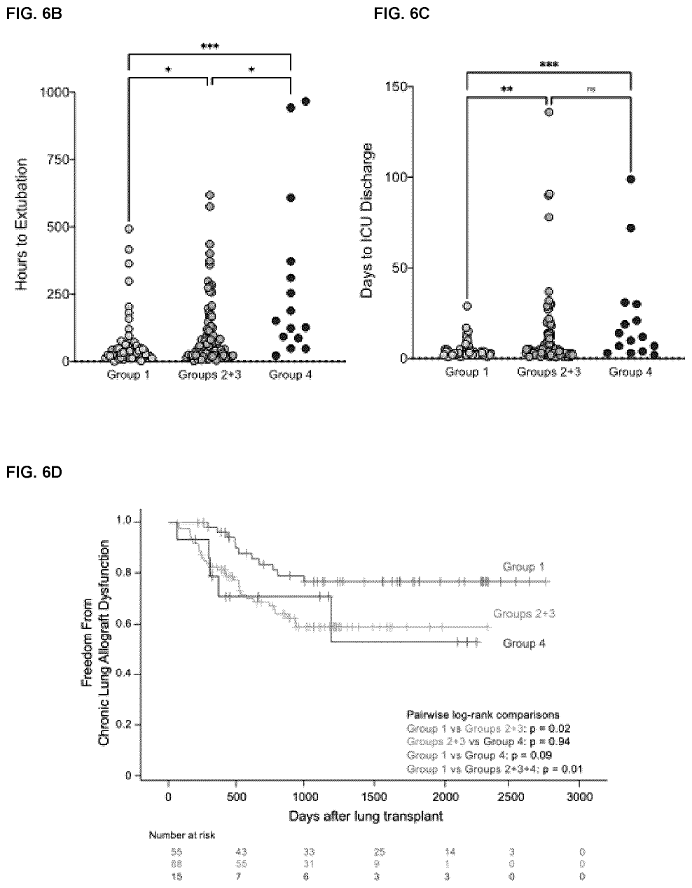

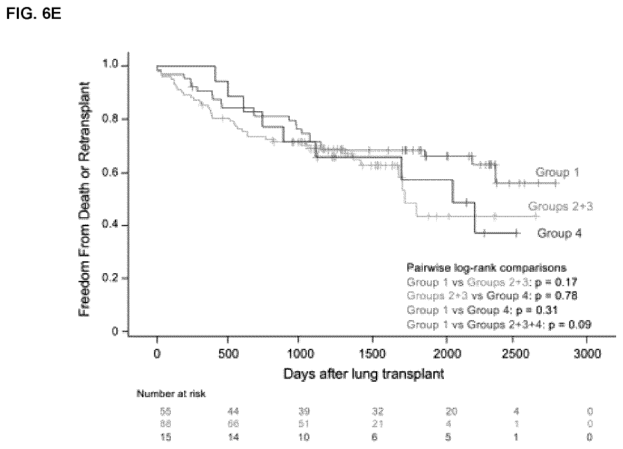

The Toronto Lung Score is a 2-plex inflammation score (TLS₂) to objectively identify ideal donor lungs by measuring IL-6 and IL-8 in EVLP perfusate (Sage et al. JHLT 2021). TLS₂ is the product of normalized IL-6 and IL-8 cytokine level in EVLP perfusate indexed from 1-100. A total of 158 donor organs in this cohort had available EVLP perfusate to measure TLS₂. Donor organs were stratified based on TBA and TLS₂ status-55 lungs had low TBA and low inflammation (Group 1), 88 lungs had either high TBA or high inflammation but not both (Group 2+3), and 15 lungs had both high TBA and high inflammation (Group 4) (FIG. 6A). Individuals who received group 1 lungs had a shorter time to extubation than those who received a Group 2+3 (p<0.05) or Group 4 (p<0.01) organ. Group 2+3 lung recipients also had a shorter time to extubation than group 4 lung recipients (p<0.05) (FIG. 6B). Similarly, group 1 lung recipients had a shorter time to ICU discharge than group 2+3 (p<0.01) and group 4 (p<0.001) lung recipients. There was no difference in time to ICU discharge between group 2+3 and group 4 lung recipients (FIG. 4C). Those who received group 1 lungs had a decreased risk of CLAD over time than those who received group 2+3 or group 4 lungs (FIG. 6D). Similarly, individuals who received group 1 lungs tended to have a decreased risk of death or needing a retransplant (FIG. 6E).

In this study, the relationship between donor LABW TBA, as a marker of aspiration, with suitability of donor lungs for transplant, performance of donor lungs on EVLP, and recipient outcomes in a large retrospective cohort was assessed. Donor TBA level >1245 nM was able to identify lungs unsuitable for transplant with a specificity of 91%. Among lungs assessed on EVLP, higher TBA level was associated with increased markers of acidemia, increased markers of inflammation, lower delta PO₂, and higher pulmonary vascular resistance. Among all transplanted donor lungs, those with high TBA levels were associated with recipient outcomes including longer time to extubation, longer time to ICU discharge, and decreased time to CLAD. Donor lungs with high TBA that were first assessed on EVLP had a shorter time to extubation than their counterparts with high TBA that were directly transplanted. Combining donor TBA with TLS₂ identified recipients with longer time to extubation, longer time to ICU discharge, and at increased risk of developing CLAD. Measuring LABW TBA at the donor hospital can help identify donor lungs most suitable for transplant.

It was observed that LABW TBA concentration was associated with increased lactate, decreased glucose, and increased markers of acidemia in EVLP perfusate. The strength of these relationships increased from the first hour to the final hour of EVLP. Upon activation, immune cells including inflammatory macrophages and effector T cells experience a Warburg-like effect resulting in increased glucose consumption and increased lactate production (McDermott et al. Cell Research 2020). Exposure of alveolar macrophages and other resident leukocytes to bile acids at time of donor death can cause activation and glycolytic flux in these immune cells. Without wishing to be bound to this theory, acute activation of resident leukocytes by exposure to bile acids at time of organ donor death can also explain the correlations observed between LABW TBA and pro-inflammatory cytokines IL-6, IL-8, and sTNFR1 in EVLP perfusate.

Bile acids can also play an indirect role in causing inflammation in donor lungs. At resting state, airway surfactants reduce surface tension and provide an innate defense barrier against environmental insults (Romero et al. Am J Respir Cell Mol Biol 2015). Certain surfactant proteins, including surfactant protein A (SP-A) and surfactant protein D (SP-D), are also signalling molecules that play an important role activating the host immune response to potentially injurious stimuli (McCormack et al. JCI 2002). Previous studies have highlighted the importance of these molecules by identifying an association between donor SP-A and SP-D loss-of-function polymorphisms with poor lung transplant recipient outcomes (Aramini et al. AJT 2013; D'Ovidio et al. ERJ 2019). Bile acids have been shown to increase phospholipase A2 (PLA2) activity leading to catabolismof airway surfactants and resultant airway inflammation (Herraez et al. J Mol Med 2014). Without wishing to be bound to this theory, aspiration of bile acids in organ donors may cause damage to donor lungs by disrupting the pulmonary lipidome which in turn can increase alveolar permeability, foam cell formation, and resultant lung inflammation/fibrosis. Alternatively, bile acids in LABW can simply be markers of aspiration and the injury could be caused by aspiration of other products from the gastrointestinal tract such as digestive enzymes and pathogen-derived products from the gut microbiome.

A strong association between donor TBA status and recipient outcomes has been identified. Interestingly, by combining donor TBA status with the Toronto Lung Score 2 (TLS₂) donor organs with low inflammation, low aspiration, and more favourable recipient outcomes were identified.

EVLP allows for further assessment and potential treatment of donor lungs before accepting the organ for transplantation. Recipient outcomes among those who received donor lungs with high TBA that were either first assessed on EVLP or directly transplanted were compared. Despite having a higher median TBA level, donor lungs with high TBA on EVLP had shorter time to extubation than their counterparts that were directly transplanted. Long-term outcomes were similar between both groups. Assessing all donor lungs with high TBA on EVLP can thus allow for improved recipient outcomes.

Demographics of donors whose lungs were used for transplant with high vs low TBA were not different. Demographics of individuals who received donor lungs with high vs low TBA concentration were not different either (Table 4). In sub-cohort with available data (n=475), inventors identified that poor recipient kidney function (eGFR<60) at time of transplant admission did not affect the relationships between donor TBA status and recipient outcomes (Table 5).

Thus, the level of bile acid in a donor level is a useful indicator for assessing the suitability of donor lungs for transplantation.

TABLE 4
Study participant demographics stratified
by donor LABW TBA level

| | TBA <1245nM | | TBA >1245nM | | |
|---|---|---|---|---|---|
| | (n = 458) | | (n = 49) | | p-value |
| Donor age | | | | | |
| Median (IQR) | 49 | (33-61) | 49 | (39-62) | 0.77 |
| Donor sex, Male, n (%) | 283 | (55) | 31 | (63) | 0.84 |
| Donor BMI (kg/m²) | | | | | |
| Median (IQR) | 26 | (23-30) | 27 | (24-30) | 0.57 |
| Donor Type, n (%) | | | | | 0.91 |
| DBD | 72 | (16) | 8 | (16) | |
| DCD | 386 | (84) | 41 | (84) | |
| Donor Cigarette Ever | 229 | (50) | 27 | (55) | 0.49 |
| Use, n (%) | | | | | |
| Recipient age at transplant | | | | | |
| Median (IQR) | 58 | (46-64) | 57 | (45-62) | 0.76 |
| Recipient sex, Male, n (%) | 267 | (58) | 33 | (67) | 0.22 |
| Recipient BMI (kg/m²) | | | | | |
| Median (IQR) | 24 | (21-26) | 24 | (22-27) | 0.83 |
| Single lung transplant, n (%) | 33 | (8) | 7 | (14) | 0.16 |
| Heart-lung transplant, n (%) | 4 | (1) | 0 | (0) | 0.51 |
| Recipient native lung | | | | | 0.94 |
| disease, n (%) | | | | | |
| Chronic obstructive | 91 | (20) | 10 | (20) | |
| pulmonary disease | | | | | |
| Cystic fibrosis | 66 | (15) | 6 | (13) | |
| Pulmonary fibrosis | 194 | (42) | 19 | (39) | |
| Other | 107 | (23) | 14 | (18) | |
| CMV mismatch | 209 | (46) | 24 | (49) | 0.66 |
| (D+/R−), n (%) | | | | | |
| Recipient urgency | | | | | 0.98 |
| status at time of | | | | | |
| transplant, n (%) * | | | | | |
| 1 | 117 | (24) | 13 | (27) | |
| 2 | 176 | (38) | 19 | (38) | |
| 3 | 161 | (35) | 17 | (35) | |
| Insufficient PFTs, n (%) | 41 | (9) | 5 | (10) | 0.77 |
| Bronchoscopy-based | | | | | |
| Characteristics | | | | | |
| Average A Score, n (%) | | | | | |
| 0 | 151 | (38) | 13 | (33) | 0.68 |
| 0.1-0.38 | 129 | (33) | 12 | (31) | |
| 0.39+ | 116 | (29) | 14 | (36) | |
| Average B Score, n (%) | | | | | |
| 0 | 289 | (93) | 29 | (83) | 0.04 |
| >0 | 23 | (7) | 6 | (17) | |
| Average Infection | | | | | |
| Score, n (%) | | | | | |
| 0 | 76 | (17) | 5 | (11) | 0.23 |
| 0.1-0.36 | 180 | (41) | 25 | (53) | |
| >0.37 | 184 | (42) | 17 | (36) | |

*Canadian urgency status: 1 is assigned to patients with lowest acuity, while 3 designates patients with highest severity of disease on the waiting list.
Abbreviations: LABW—Large Airway Bronchial Wash, TBA—Total Bile Acid, BMI—Body Mass Index, DBD—Donation before brain death, DCD—Donation before cardiac death, CMV—Cytomegalovirus, PFT—Pulmonary function test

TABLE 5
Cox-PH models assessing the impact of recipient kidney function
on the relationship between donor LABW TBA status and recipient
outcomes in a sub-cohort post-hoc analysis (n = 475)

| | |
|---|---|
| HR (95% CI) | p-value |

Primary graft dysfunction grade 3 at 72 hours post-transplant

| | | |
|---|---|---|
| TBA >1245 nM | 2.45 (1.28-4.13) | <0.001 |
| eGFR <60 | 1.11 (0.79-1.55) | 0.53 |
| Multivariable: TBA + eGFR | 2.45 (1.29-4.13) | <0.001 |
| Multivariable: TBA + eGFR + | 2.43 (1.30-4.52) | <0.001 |
| covariates | | |
| Multivariable: TBA + | 2.42 (1.31-4.49) | <0.001 |
| covariates only | | |

Time to extubation

| | | |
|---|---|---|
| TBA >1245 nM | 0.45 (0.27-0.74) | 0.002 |
| eGFR <60 | 0.87 (0.62-1.22) | 0.42 |
| Multivariable: TBA + eGFR | 0.46 (0.25-0.77) | 0.004 |
| Multivariable: TBA + eGFR + | 0.41 (0.28-0.56) | <0.001 |
| covariates | | |
| Multivariable: TBA + | 0.39 (0.29-0.55) | <0.001 |
| covariates only | | |

ICU length of stay

| | | |
|---|---|---|
| TBA >1245 nM | 0.48 (0.35-0.66) | <0.001 |
| eGFR <60 | 1.20 (0.73-1.84) | 0.77 |
| Multivariable: TBA + eGFR | 0.49 (0.37-0.67) | <0.001 |
| Multivariable: TBA + eGFR + | 0.45 (0.27-0.65) | <0.001 |
| covariates | | |
| Multivariable: TBA + | 0.42 (0.28-0.63) | <0.001 |
| covariates only | | |

Time to chronic lung allograft dysfunction

| | | |
|---|---|---|
| TBA >1245 nM | 1.90 (1.28-2.82) | 0.001 |
| eGFR <60 | 0.86 (0.47-1.57) | 0.63 |
| Multivariable: TBA + eGFR | 2.08 (1.38-3.13) | <0.001 |
| Multivariable: TBA + eGFR + | 1.63 (1.02-2.62) | 0.04 |
| covariates | | |
| Multivariable: TBA + | 1.64 (1.03-2.63) | 0.04 |
| covariates only | | |

Time to death or retransplantation

| | | |
|---|---|---|
| TBA >1245 nM | 1.25 (0.90-2.22) | 0.25 |
| eGFR <60 | 1.18 (0.77-1.81) | 0.45 |
| Multivariable: TBA + eGFR | 1.26 (0.87-2.34) | 0.27 |
| Multivariable: TBA + eGFR + | 1.46 (0.99-2.18) | 0.18 |
| covariates | | |
| Multivariable: TBA + | 1.43 (0.96-2.14) | 0.12 |
| covariates only | | |

Abbreviations: LABW—Large Airway Bronchial Wash, TBA—Total Bile Acid, eGFR—estimated glomerular filtration rate, ICU—intensive care unit
Example 2 - Electrochemical Bile Acid Assay

Electrochemical measurements were performed on a Bioanalytical Systems (BASi) epsilon potentiostat with a three-electrode system featuring an Ag/AgCl reference electrode (BASi), a platinum wire auxiliary electrode, and a biosensing electrode serving as the working electrode. Electrodes were incubated in a solution of NAD+/NADH, 3α-HSD and lung wash sample, then scanned using differential pulse voltammetry (DPV).

Electrochemical measurements are useful in performing the methods described herein in measuring the levels of bile acids for assessing the suitability of donor lungs for transplantation.

### Example 3—Detection of Bile Acids Using Enzymatic Cycling TBA Assay (Fifth Generation Bile Acids Test)

Enzymatic colorimetric technique used for quantitively assess the level of total bile acids in biological samples. The enzyme 3-α-hydroxysteroid dehydrogenase (3-α-HSD) converts bile acids into Thio-NADH and 3-keto steroids in the presence of Thio-NAD. The rate of formation of Thio-NADH can be determined by measuring absorbance at 405 nm, which can be used to quantify the amount of total bile acids within the LABW sample.

Enzymatic colorimetric technique is useful in performing the methods described herein in measuring the levels of bile acids for assessing the suitability of donor lungs for transplantation.

### Example 4A—Enzyme-Linked Immunosorbent Assay (ELISA) for Detecting Bile Acids

Antibody-based detection of specific bile acids components such as TCA and GCA (MyBioSource ELISA kit) to quantitively assess the level of bile acids in donor LABW. The assay is based on detecting antigens of interest (e.g., TCA and GCA) in LABW samples using antibodies that specifically bind to these antigens. The presence of antibodies can be quantitively assessed using the visible signals (absorbance) produced by enzymes linked to the antibodies.

ELISA can also be part of a point-of-care device such as a lateral flow device. One example of the lateral flow device is a simple and easy to use diagnostic kit with a sandwich ELISA lateral flow format for health care professional in a lung transplant team. In brief, the samples first come in touch with colored particles labeled with antibody raised against one or more bile acids (target analyte). The antibody can be used in the test line. The test line can also show a colorful band. The intensity of this band correlates with bile acid levels. This simple kit can come with a band color intensity meter to assist the judgment on bile acid levels.

The second would be a quantitative device: In this device the intensity of the test line can be measured to determine the quantity of bile acids in the sample. A lateral flow reader (for example, a handheld diagnostic device) can be used to provide a fully quantitative assay result. Using a suitable detection technology (complementary metal-oxide semiconductor (CMOS) or charge coupled device (CCD)) and unique wavelength of light for illumination, one can obtain a signal rich image from the actual test lines. With an image processing algorithm specifically designed for a bile acids ELISA measurement, line intensities can then be converted to bile acids concentrations. This quantitative lateral flow device can be used in the field hospitals and clinics, as well as diagnostic medical laboratories.

The same principles apply for a lateral flow device for detecting inflammation markers.

### Example 4B—Enzyme-Linked Immunosorbent Assay (ELISA) for Detecting Total Bile Acids (TBA) in Lung Donor Bronchial Washings as a Marker of Aspiration and Predictor of Lung Transplant Recipient Outcomes

The utility of a commercially available TBA ELISA was tested to detect aspiration and predict recipient outcomes in lung donors. Inventors measured TBA using a human competitive TBA ELISA (MyBioSource cat no: MBS723419). This TBA ELISA kit applies the competitive enzyme immunoassay technique using a polyclonal anti-TBA antibody and an TBA-HRP conjugate. The lung donor bronchial washings samples and buffer were incubated together with TBA-HRP conjugate in pre-coated plate for one hour. Following the incubation period, the wells were decanted and washed five times, and then the wells were incubated with a substrate for HRP enzyme. The product of the enzyme-substrate reaction formed a blue colored complex. To stop the reaction. a stop solution was added, which turned the solution yellow. The intensity of color was measured spectrophotometrically at 450 nm in a microplate reader. The intensity of the color was inversely proportional to the TBA concentration since TBA from samples and TBA-HRP conjugate competed for the anti-TBA antibody binding site. Since the number of sites were limited, as more sites were occupied by TBA from the lung donor bronchial washings samples, fewer sites were left to bind TBA-HRP conjugate. A standard curve was plotted relating the intensity of the color (O.D.) to the concentration of standards. The TBA concentration in each sample was interpolated from this standard curve.

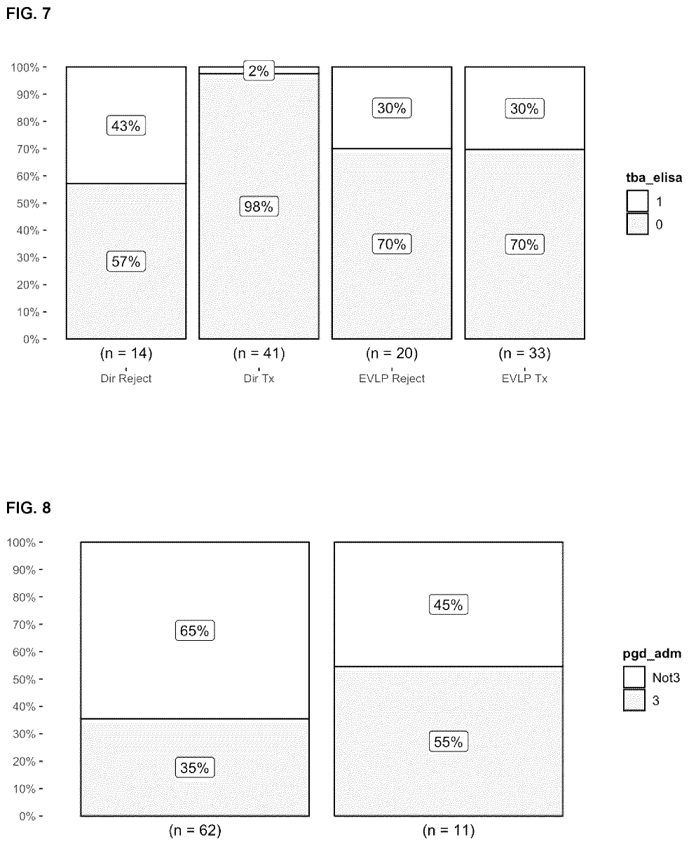

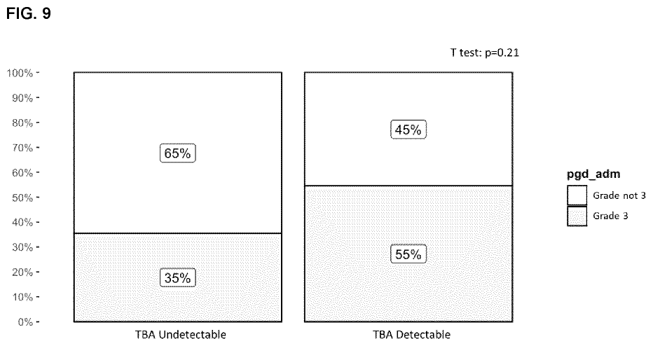

Cohort in this Example included consecutive lung donors at the Toronto Lung Transplant Program from 2012-2018 with available excess BW samples (n=73). The results shows that TBA was detectable in 11/73 donor BW samples in this cohort. Downstream analysis compared lung donors with BW samples that had detectable vs undetectable TBA. In particular, FIG. 7 shows differences in groups TBA was detectable in 43% of lungs that were not accepted for transplant, 2% of lungs that were directly transplanted, and 30% of lungs that were further assessed on EVLP (p-value<0.001). FIG. 8 shows results relating to primary graft dysfunction (PGD) at admission, which indicate a trend towards more prevalent PGD3 in patients with detectable TBA. FIG. 9 shows primary graft dysfunction (PGD) at 72 h post-operation, which indicates a trend towards more prevalent PGD3 in patients with detectable TBA. Table 3 below shows donor TBA and recipient outcomes. Table 3 shows that detectable TBA was associated with time to death/retransplantation, which indicate trends towards being associated with time to extubation and time to ICU discharge.

TABLE 3
Donor TBA and Recipient Outcomes:

Univariable Cox-PH

| | |
|---|---|
| HR (95% CI) | p-value |

Time to extubation

| | | |
|---|---|---|
| TBA detectable with ELISA | 0.57 (0.29-1.14) | 0.11 |

Time to ICU discharge

| | | |
|---|---|---|
| TBA detectable with ELISA | 0.56 (0.28-1.11) | 0.09 |

Time to CLAD

| | | |
|---|---|---|
| TBA detectable with ELISA | 1.61 (0.55-4.71) | 0.38 |

Time to death/retransplantation

| | | |
|---|---|---|
| TBA detectable with ELISA | 2.50 (1.22-5.09) | 0.01 |

Together, these results showed that ELISA tests can be used in measuring the levels of TBA in assessing suitability of donor lungs in transplantation. Thus, POC devices, including lateral flow devices, that incorporate ELISA can be useful in performing the methods described herein in measuring the levels of bile acids and/or inflammation markers for assessing the suitability of donor lungs for transplantation.

### Example 5—Detection of Bile Acids and Inflammation Markers Using Mass Spectrometry

Levels of bile acids and inflammation markers was quantitatively determined based on the mass-to-charge ratio of the molecules within the BAL/LABW samples. The chemical compounds are ionized, accelerated, separated and detected based on their mass-to-charge ratio. The resultant spectrum allows identification and quantification of compounds of interest, including bile acids such as taurocholic acid (TCA), glycocholic acid (GCA) and cholic acid (CA), and inflammation markers such as interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10).

Mass spectrometry for detection of bile acids is approximately a thousand-fold more sensitive than the more commonly used calorimetric total bile acid assay. Mass spectrometry allowed for quantification of individual bile acid species, in contrast to common BAL reports which measured total bile acid. The skilled person recognizes that any suitable mass spectrometric techniques, such as liquid chromatography-tandem mass spectrometry, can be used to quantitively assess the levels of bile acids and inflammation markers. Mass spectrometry is useful in performing the methods described herein in measuring the levels of bile acids and/or inflammation markers for assessing the suitability of donor lungs for transplantation.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS FOR REFERENCES REFERRED TO IN THE SPECIFICATION

1. M. Cypel, J. C. Yeung, S. Keshavjee, Novel approaches to expanding the lung donor pool: donation after cardiac death and ex vivo conditioning. Clin Chest Med 32, 233-244 (2011).
2. M. K. Hsin, I. Iskender, D. Nakajima, M. Chen, H. Kim, P. R. dos Santos, J. Sakamoto, J. Lee, K. Hashimoto, C. Harmantas, D. Hwang, T. Waddell, M. Liu, S. Keshavjee, M. Cypel, Extension of donor lung preservation with hypothermic storage after normothermic ex vivo lung perfusion. J Heart Lung Transplant 35, 130-136 (2016).
3. T. N. Machuca, O. Mercier, S. Collaud, J. Tikkanen, T. Krueger, J. C. Yeung, M. Chen, S. Azad, L. Singer, K. Yasufuku, M. de Perrot, A. Pierre, T. K. Waddell, S. Keshavjee, M. Cypel, Lung transplantation with donation after circulatory determination of death donors and the impact of ex vivo lung perfusion. Am J Transplant 15, 993-1002 (2015).
4. D. Nakajima, M. Cypel, T. Martinu, M. Liu, S. Keshavjee, Donor bronchial wash bile acid and suitability of donor lungs for transplantation. J Heart Lung Transplant 37, 304-306 (2017).
5. D. Nakajima, M. Liu, A. Ohsumi, R. Kalaf, I. Iskender, M. Hsin, T. Kanou, M. Chen, B. Baer, R. Coutinho, L. Maahs, P. Behrens, S. Azad, T. Martinu, T. K. Waddell, J. F. Lewis, M. Post, R. A. W. Veldhuizen, M. Cypel, S. Keshavjee. Lung lavage and surfactant replacement during ex vivo lung perfusion for treatment of gastric acid aspiration-induced donor lung injury. J Heart Lung Transplant 36, 577-585 (2017).
6. M. Cypel, S. Keshavjee, The clinical potential of ex vivo lung perfusion. Expert Rev Respir Med 6, 27-35 (2012).
7. M. Cypel, M. Rubacha, J. Yeung, S. Hirayama, K. Torbicki, M. Madonik, S. Fischer, D. Hwang, A. Pierre, T. K. Waddell, M. de Perrot, M. Liu, S. Keshavjee, Normothermic ex vivo perfusion prevents lung injury compared to extended cold preservation for transplantation. Am J Transplant 9, 2262-2269 (2009)
8. M. Cypel, J. C. Yeung, S. Hirayama, M. Rubacha, S. Fischer, M. Anraku, M. Sato, S. Harwood, A. Pierre, T. K. Waddell, M. de Perrot, M. Liu, S. Keshavjee, Technique for prolonged normothermic ex vivo lung perfusion. J Heart Lung Transplant 27, 1319-1325 (2008).
9. M. Cypel, J. C. Yeung, M. Liu, M. Anraku, F. Chen, W. Karolak, M. Sato, J. Laratta, S. Azad, M. Madonik, C. W. Chow, C. Chaparro, M. Hutcheon, L. G. Singer, A. S. Slutsky, K. Yasufuku, M. de Perrot, A. F. Pierre, T. K. Waddell, S. Keshavjee, Normothermic ex vivo lung perfusion in clinical lung transplantation. N Engl J Med 364, 1431-1440 (2011).
10. J. C. Yeung, M. Cypel, E. Massad, S. Keshavjee, Ex vivo lung perfusion and reconditioning. Multimed Man Cardiothorac Surg 2011, mmcts 2009 004242 (2011).
11. Kennedy, V. E., Todd, J. L. & Palmer, S. M. Bronchoalveolar Lavage as a Tool to Predict, Diagnose and Understand Bronchiolitis Obliterans Syndrome: Bronchoalveolar Lavage in BOS. American Journal of Transplantation 13, 552-561 (2013).
12. Young, L. R., Hadjiliadis, D., Davis, R. D. & Palmer, S. M. Lung Transplantation Exacerbates Gastroesophageal Reflux Disease*. Chest 124, 1689-1693 (2003).
13. Hathorn, K. E., Chan, W. W. & Lo, W.-K. Role of gastroesophageal reflux disease in lung transplantation. WJT 7, 103 (2017).
14. King, B. J., Iyer, H., Leidi, A. A. & Carby, M. R. Gastroesophageal Reflux in Bronchiolitis Obliterans Syndrome: A New Perspective. The Journal of Heart and Lung Transplantation 28, 870-875 (2009).
15. Lawenko, R. M. A. & Lee, Y. Y. Evaluation of Gastroesophageal Reflux Disease Using the Bravo Capsule pH System. J Neurogastroenterol Motil 22, 25-30 (2015).

1. A method of assessing significant aspiration in a donor lung, the method comprising:
a) obtaining a bronchial wash sample that has been obtained from the donor lung;
b) measuring a level of a bile acid in the bronchial wash sample;
c) comparing the level of the bile acid with a bile acid cut-off level;
d) identifying the donor lung as likely to have had significant aspiration when the level of the bile acid is greater than the bile acid cut-off level or unlikely to have had significant aspiration when the bile acid level is equal to or less than the bile acid cut-off level, optionally wherein the bile acid cut-off level is determined by calculating in a population of donor lungs an average bile acid cut-off level above which at least 90% of the donor lungs were identified as having significant aspiration, and
e) optionally, i) when the level of the bile acid is greater than the bile acid cut-off level, the method described herein further comprises performing EVLP to confirm lung function prior to transplantation; ii) when the level of the bile acid is greater than the bile acid cut-off level, the method further comprises treating the lungs for aspiration damage, optionally treating the lungs with surfactant; iii) when the level of the bile acid is greater than the bile acid cut-off level, the method further comprises rejecting the lung for transplantation.

2. The method of claim 1, wherein the bronchial wash sample is a bronchoalveolar lavage (BAL) sample or a large airway bronchial wash (LABW) sample

3. The method of claim 1 or 2, wherein the bile acid is total bile acid (TBA) or a component thereof optionally selected from taurocholic acid (TCA), glycocholic acid (GCA), cholic acid (CA), and combinations thereof.

4. The method of claim 1 or 2, wherein the bile acid is TBA.

5. The method of claim 4, wherein the cut-off level is about 1245 nM and a level of the TBA greater than the cut-off level is indicative the donor lung as likely to have significant aspiration and a level of TBA equal to or less than the cut-off level is indicative the donor lung is unlikely to have had significant aspiration, wherein the cut-off level is calculated by instilling and suctioning 20 ml of normal saline.

6. The method of any one of claims 1 to 5, wherein the method is carried out at point-of-care.

7. A method of assessing suitability of a donor lung for transplant, the method comprising:
a) obtaining a bronchial wash sample that has been obtained from the donor lung;
b) measuring a level of a bile acid in the bronchial wash sample and optionally a level of one or more inflammation markers;
c) comparing the level of the bile acid with a bile acid cut-off level;
d) optionally comparing the level of the one or more inflammation markers with an inflammation marker cut-off level;
e) identifying the donor lung as not suitable for transplant when the level of the bile acid is greater than the bile acid cut-off level, and optionally when the level of the one or more inflammation markers is greater than the inflammation marker cut-off level, or as suitable for transplant when the level of the bile acid is equal or less than the bile acid cut-off level, and optionally when the level of the one or more inflammation markers is equal or less than the inflammation marker cut-off level, optionally wherein the bile acid cut-off level is determined by calculating in a population of donor lungs an average bile acid cut-off level above which at least 90% of the donor lungs were identified as not suitable for transplantation and the inflammation marker cut-off level is determined by calculating in a population of donor lungs an average inflammation marker cut-off level above which at least 90% of the donor lungs were identified as not suitable for transplantation; and
f) optionally, i) when the level of the bile acid is greater than the bile acid cut-off level, the method described herein further comprises performing EVLP to confirm lung function prior to transplantation; ii) when the level of the bile acid is greater than the bile acid cut-off level, the method further comprises treating the lungs for aspiration damage, optionally treating the lungs with surfactant; iii) when the level of the bile acid is greater than the bile acid cut-off level, the method further comprises rejecting the lung for transplantation.

8. The method of claim 7, wherein the bronchial wash sample is a bronchoalveolar lavage (BAL) sample or a large airway bronchial wash (LABW) sample.

9. The method of claim 7 or 8, wherein the bile acid is total bile acid (TBA) or a component thereof optionally selected from taurocholic acid (TCA), glycocholic acid (GCA), cholic acid (CA), and combinations thereof.

10. The method of claim 7 or 8, wherein the bile acid is TBA.

11. The method of claim 10, wherein the cut-off level is about 1245 nM and a level of the TBA greater than the cut-off level is indicative the donor lung is not suitable for transplantation and a level of TBA equal to or less than the cut-off level is indicative the donor lung is suitable for transplantation, wherein the cut-off level is calculated by instilling and suctioning 20 ml of normal saline.

12. The method of claim 10, wherein the one or more inflammation markers are measured and are selected from interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10).

13. The method of claim 10, wherein the one or more inflammation markers is IL-6.

14. The method of claim 10, wherein the one or more inflammation markers is IL-8.

15. The method of any one of claims 1 to 14, wherein the method is carried out at point-of-care.

16. The method of any one of claims 1 to 15, wherein when the donor lung identified as suitable for transplant, the donor lung is retrieved from the donor and is directly transplanted to a recipient or is subjected to ex vivo lung perfusion (EVLP).

17. The method of any one of claims 1 to 15, wherein when the donor lung is identified as not suitable for transplant, the donor lung is declined and/or not retrieved from the donor.

18. The method of any one of claims 1 to 17, wherein the donor lung being assessed is EVLP naïve.

19. The method of any one of claims 1 to 15, wherein the donor lung being assessed for suitability for transplant is undergoing EVLP.

20. The method of any one of claims 1 to 19, wherein the level of the bile acid and optionally the level of the one or more inflammation markers are detected or determined by Luminex® based assays, Western blots, immunoassay e.g. ELISA, immunofluorescence, radioimmunoassay, dot blotting, FACS, protein microarray, immunoprecipitation followed by SDS-PAGE, immunocytochemistry, simple Plex assay, multiplex assay, mass spectrometry, electrochemical assay, enzymatic assay or colorimetric assay.

21. A method of assessing the likelihood of developing gastroesophageal reflux disease (GERD) in a donor lung recipient, the method comprising:
a) obtaining a bronchial wash sample from the donor lung recipient;
b) measuring a level of a bile acid in the bronchial wash sample;
c) comparing the level of the bile acid with a bile acid cut-off level;
d) identifying the donor lung recipient as likely to develop GERD when the level of the bile acid is greater than the bile acid cut-off level, or unlikely to develop GERD when the bile acid level is equal to or less than the bile acid cut-off level, optionally wherein the bile acid cut-off level is determined by calculating in a population of donor lungs an average bile acid cut-off level above which at least 90% of the donor lungs were identified as having developed GERD; and
e) optionally, i) when the level of the bile acid is greater than the bile acid cut-off level, the method described herein further comprises performing EVLP to confirm lung function prior to transplantation; ii) when the level of the bile acid is greater than the bile acid cut-off level, the method further comprises treating the lungs for aspiration damage, optionally treating the lungs with surfactant; iii) when the level of the bile acid is greater than the bile acid cut-off level, the method further comprises rejecting the lung for transplantation.

22. The method of claim 21, wherein the bronchial wash sample is a bronchoalveolar lavage (BAL) sample or a large airway bronchial wash (LABW) sample

23. The method of claim 21 or 22, wherein the bile acid is total bile acid (TBA) or a component thereof optionally selected from taurocholic acid (TCA), glycocholic acid (GCA), cholic acid (CA), and combinations thereof.

24. The method of claim 23, wherein the bile acid is TBA.

25. The method of claim 24, wherein the cut-off level is about 1245 nM and a level of the TBA greater than the cut-off level is indicative the donor lung as likely to develop GERD and a level of TBA equal to or less than the cut-off level is indicative the donor lung as unlikely to develop GERD, wherein the cut-off level is calculated by instilling and suctioning 20 ml of normal saline.

26. The method of any one of claims 1 to 25, wherein the level of the bile acid and optionally the level of the one or more inflammation markers are detected or determined by Luminex® based assays, Western blots, immunoassay e.g. ELISA, immunofluorescence, radioimmunoassay, dot blotting, FACS, protein microarray, immunoprecipitation followed by SDS-PAGE, immunocytochemistry, simple Plex assay, multiplex assay, mass spectrometry, electrochemical measurements, enzymatic assay or colorimetric assay.

27. A kit comprising at least one detection agent specific for a biomarker selected from bile acid, optionally total bile acid, taurocholic acid (TCA), glycocholic acid (GCA) or cholic acid (CA), and optionally an inflammation marker, optionally interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10), the kit optionally further comprising one or more of a 96-well plate, standards, assay buffer, wash buffer, sample diluent, standard diluent, detection antibody diluent, streptavidin-PE, a filter plate and sealing tape, optionally for performing the method of any one of claims 1 to 26.

28. A point-of-care (POC) device for detecting the presence of bile acid in a sample, comprising:
a housing,
a porous medium comprised in the housing, the porous medium comprising at a first end a sample portion and at a second end a testing portion,
a channel defined between the sample portion and the testing portion, the channel defining a flow path along which the sample passes and is drawn from the sample portion to the testing portion by capillary action, and
one or more reagents or detection agents for bile acid, optionally in fluid communication with the testing portion or immobilized on the testing portion, the one or more reagents or detection agents being capable of reacting with the bile acid to generate a detection signal, optionally a visible color change or a fluorescent signal.

29. The device of claim 28, comprising a plurality of flow paths through which the sample passes and which distributes the sample to a plurality of testing zones within the testing portion.

30. The device of claim 29, wherein each of the testing zones comprises a different reagent or detection agent.

31. The device of any one of claims 28 to 30, wherein the bile acid is selected from total bile acid (TBA), taurocholic acid (TCA), glycocholic acid (GCA), cholic acid (CA), and combinations thereof.

32. The device of any one of claims 28 to 31, further comprising one or more reagents or detection agents for inflammation markers, optionally selected from interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10).

33. The device of any one of claims 28 to 32, wherein the presence of bile acid and optionally the one or more inflammation marker is detected using immunoassay, optionally enzyme-linked immunosorbent assay (ELISA), a multiplex assay, electrochemical assay, enzymatic assay, colorimetric assay and/or mass spectrometry.

34. The device of claim any one of claims 28 to 33, wherein the device is a lateral flow device.

35. The device of any one of claims 28 to 34, wherein the fluorescent signal is detected by a reader.

36. A point-of-care (POC) device for performing the method of any one of claims 1 to 26, comprising:
a housing,
a porous medium comprised in the housing, the porous medium comprising at a first end a sample portion and at a second end a testing portion,
a channel defined between the sample portion and the testing portion, the channel defining a flow path along which the sample passes and is drawn from the sample portion to the testing portion by capillary action, and
one or more reagents or detection agents for bile acid, optionally in fluid communication with the testing portion or immobilized on the testing portion, the one or more reagents or detection agents being capable of reacting with the bile acid to generate a detection signal, optionally a visible color change or a fluorescent signal.

37. The device of claim 36, comprising a plurality of flow paths through which the sample passes and which distributes the sample to a plurality of testing zones within the testing portion.

38. The device of claim 37, wherein each of the testing zones comprises a different reagent or detection agent.

39. The device of any one of claims 36 to 38, wherein the bile acid is selected from total bile acid (TBA), taurocholic acid (TCA), glycocholic acid (GCA), cholic acid (CA), and combinations thereof.

40. The device of any one of claims 36 to 39, further comprising one or more reagents or detection agents for inflammation markers, optionally selected from interleukin-6 (IL-6), interleukin-8 (IL-8), interleukin-1alpha (IL-1α), interleulkin-1beta (IL-1β), C—C motif chemokine ligand 2 (CCL2), C—C motif chemokine ligand 5 (CCL5), C—X—C motif chemokine ligand 9 (CXCL9) and C—X—C motif chemokine ligand 10 (CXCL10).

41. The device of any one of claims 36 to 40, wherein the presence of bile acid and optionally the one or more inflammation marker is detected using immunoassay, optionally enzyme-linked immunosorbent assay (ELISA), a multiplex assay, electrochemical assay, enzymatic assay, colorimetric assay and/or mass spectrometry.

42. The device of any one of claims 36 to 41, wherein the device is a lateral flow device.

43. The device of any one of claims 36 to 42, wherein the fluorescent signal is detected by a reader.
